(19) United States
(12) Patent Application Publication
(10) Pub. No.: US 2026/0103673 A1
(43) Pub. Date: Apr. 16, 2026

(54) MICROBIAL COMPOSITIONS AND METHODS OF PREPARING SAME AND USE THEREOF

(71) Applicant: MYBIOTICS PHARMA LTD., Rehovot (IL)

(72) Inventors: David DABOUSH, Mishmar David (IL); Lior WEISSMAN, Ness-Ziona (IL); Itay KILOVATY, Rehovot (IL); Igor LEV, Frankfurt am Main (DE); Alon SHTRIKMAN, Jerusalem (IL); Dorit ELI, Petah Tikva (IL)

(21) Appl. No.: 19/114,838

(22) PCT Filed: Sep. 25, 2022
(86) PCT No.: PCT/IL2022/051020

§ 371 Date: Mar. 25, 2025

(51) Int. Cl. C12N 1/20; C12N 1/02; C12N 11/00; C12Q 1/02; C12Q 1/04; C12Q 1/6869

(52) U.S. Cl. CPC C12N 1/20; C12N 1/02; C12N 11/00; C12Q 1/025; C12Q 1/04; C12Q 1/6869

(57) ABSTRACT

The present invention, in some embodiments, is directed to a method for producing a composition including a co-culture comprising a plurality of bacteria having a high similarity % to an origin sample and bacterial load.

### CROSS REFERENCE TO RELATED APPLICATIONS

This application claims the benefit of priority of U.S. Provisional Patent Application No. 63/292,536, filed on Dec. 22, 2021, titled “SCREENING SYSTEMS AND ASSAYS USING BACTERIA and Israeli Patent Application No. 286748, filed on Sep. 27, 2021, titled “MICROBIAL COMPOSITIONS AND METHODS OF PREPARING SAME”, the contents of which are incorporated herein by reference in their entirety.

### FIELD OF THE INVENTION

The present invention is in the field of microbiology, and particularly relates to complex microbial compositions, and methods for preparing and using same.

### BACKGROUND

There is a growing body of evidence advocating the use of probiotics to promote human health, e.g., benefiting the immune system, suppressing infections, etc.

The human body contains bacterial cells, creating a network of bacterial-human cell interactions that greatly affect our health status and even our behavioral patterns. For example, the gastrointestinal tract harbors an abundant and diverse microbial community. It is a complex system, providing an environment or niche for a community of many different microorganisms, including diverse species of bacteria. Microbial flora also inhabits other body areas such as skin, nails, eyes, oral and upper respiratory tract and urogenital tract.

A healthy microbiota includes bacterial colonization by a balanced community which provides the host with multiple benefits including resistance to a broad spectrum of pathogens, production of and absorption of essential nutrients, and an appropriately controlled systemic immunity. In settings of ‘dysbiosis’ or disrupted symbiosis, microbiota functions can be lost or deranged, resulting in increased susceptibility to pathogens, altered metabolic profiles, or induction of proinflammatory signals that can result in local or systemic inflammation or autoimmunity. Thus, microbial flora, including intestinal microbiota, is considered a key player in the pathogenesis of many diseases and disorders, including but not limited to, a variety of pathogenic infections of the gut.

Current practice of treating gut dysbiosis includes fecal transplantation from healthy donors. Typically, fecal transplantation, also called fecal microbiota transplantation (FMT), is a method for re-establishment and/or modulation of a recipient's gastrointestinal tract microbiota, with the aim of preventing, treating and/or ameliorating a disease or condition. Research show that FMT can restore health-associated bacteria in the lower intestine, thereby treating, e.g., Clostridioides difficile (C. diff) infection and prevent its recurrence (recurrent C. diff, rCDI). C. diff is one of the causes of healthcare-associated diarrhea due to a severely disrupted microbiome, e.g., due to exacerbated use of antibiotics. According to systematic searches [Gupta K, Tappiti M, Nazir A M, et al. (May 5, 2022) Fecal Microbiota Transplant in Recurrent Clostridium Difficile Infections: A Systematic Review. Cureus 14(5): e24754. doi:10.7759/cureus.24754], FMT has demonstrated more than 90% cure rate among C. diff patients who underwent several FMT treatments. Although FMT is rapidly becoming an accepted treatment for many other diseases, linked to a disrupted intestinal microbiome [Baktash A, Terveer E M, Zwittink R D, Hornung B V H, Corver J, Kuijper E J and Smits W K (2018) Mechanistic Insights in the Success of Fecal Microbiota Transplants for the Treatment of Clostridium difficile Infections. Front. Microbiol. 9:1242. doi: 10.3389/fmicb.2018.01242], there are concerns around its safety, low reproducibility, and manufacturing scalability.

There remains a great need for methods for preparing bacterial compositions having increased similarity to an origin sample, e.g., a fecal sample, which may be used for treating dysbiosis.

Advantageously, such methods may reduce the need for the redundant collection of an origin sample, reduce safety risks associated with the use of a biological sample, and preserve the original microbiota profile while allowing manufacturing scalability and reproducibility.

Such composition can also be used inter-alia as a system that simulates an in-vitro microbial environmental niche of a subject, e.g., gastrointestinal flora of a subject, thus capable of providing insight to bacterial population modulation in view of an exposure to at least one compound.

### SUMMARY

In some aspects of the invention, there is provided a method for producing a composition comprising a co-culture comprising a plurality of bacteria grown in the presence of a particle, the co-culture comprises: i) at least 30% similarity to an origin sample, and ii) a bacterial load of at least 1·E⁴ per 1 gr particle.

In some aspects of the invention, there is provided a composition produced according to the method of the invention.

In some aspects of the invention, there is provided an in-vitro method for evaluating the effect of: at least one compound on a plurality of bacteria, a plurality of bacteria on at least one compound, or both.

In some embodiments, the method comprises the steps of: providing microorganisms comprising a plurality of bacteria being derived from an origin sample; contacting said plurality of bacteria with a particle, and allowing said plurality of bacteria to at least partially attach to said particle; and culturing said plurality of bacteria at least partially attached to said particle in a growth medium for a period of less than 14 days, wherein said culturing comprises culturing under anaerobic conditions, thereby producing the composition comprising a co-culture comprising a plurality of bacteria grown in the presence of a particle comprising: i) at least 30% similarity to an origin sample; and ii) a bacterial load of at least 1·E⁴ per 1 gr particle.

In some embodiments, the method comprises the steps of: providing a plurality of bacteria being derived from an origin sample; contacting said plurality of bacteria with a particle, and allowing said plurality of bacteria to at least partially attach to said particle; and culturing said plurality of bacteria at least partially attached to said particle in a growth medium for a period of less than 14 days, wherein said culturing comprises culturing under anaerobic conditions, thereby producing the composition comprising a co-culture comprising a plurality of bacteria grown in the presence of a particle comprising: i) at least 30% similarity to an origin sample; and ii) a bacterial load of at least 1·E⁴ per 1 gr particle.

In some embodiments, the plurality of bacteria are characterized as having differing growth, cultivation and/or proliferative conditions selected from the group consisting of: metabolic requirements, nutritional requirements, pH, Temperature, aerobic, obligatory anaerobic, facultative anaerobic, microaerophilic, attached form, planktonic, growth medium, flow, shake, stir, agitation, static, moist, low-humidity, and any combination thereof.

In some embodiments, the culturing is carried out until said co-culture reaches a similarity greater than or equal to 30% to said origin sample, and a bacterial load of at least 1·E⁴ per 1 gr particle.

In some embodiments, the culturing period ranges from 6 hours to 14 days.

In some embodiments, the co-culture comprises at least 30% similarity to the origin sample when said similarity is determined by a metric that considers the genetic relatedness of the bacteria using any one of: next generation sequencing (NGS) technology, whole genome sequencing (WGS), or both.

In some embodiments, the similarity comprises at least 50% Weighted Unifrac similarity. In some embodiments, the co-culture comprises at least 50% Weighted Unifrac similarity.

In some embodiments, the co-culture comprises at least 70% similarity to the origin sample, and a bacterial load of at least 1·E⁸ per 1 gr particle.

In some embodiments, the similarity comprises at least 70% Weighted Unifrac similarity, and said co-culture comprises a bacterial load of at least 1·E⁸ per 1 gr particle.

In some embodiments, the said similarity comprises any one of: presence of a bacterial taxonomic classes, genetic relatedness, phylogenetic distances between observed bacteria, bacterial diversity, abundance of bacteria, relative abundance, or any combination thereof.

In some embodiments, the similarity between said co-culture and said origin sample comprises similarity between bacterial populations.

In some embodiments, the origin sample further comprises additional microorganisms comprising any one of: archaea, viruses, fungi, or any combination thereof.

In some embodiments, the additional microorganisms are contacted with the particle.

In some embodiments, the additional microorganisms are at least partially attached to the particles.

In some embodiments, the co-culture further comprises said additional microorganisms.

In some embodiments, the similarity between said co-culture and said origin sample further comprises similarity of at least one of: archaea, viruses, fungi populations, or any combination thereof.

In some embodiments, the provided plurality of bacteria belong to at least 5 species of bacteria and/or at least 2 bacterial genera.

In some embodiments, the composition comprises bacteria in planktonic form, and bacteria at least partially attached to said particles.

In some embodiments, said growth medium comprises at least two carbon sources.

In some embodiments, the growth medium comprises carbon sources from at least two chemical groups being selected from the group consisting of: monosaccharide, di-saccharide, polysaccharide and any combination thereof.

In some embodiments, the growth medium comprises at least one monosaccharide, at least one disaccharide and at least one polysaccharide.

In some embodiments, the contacting step, the culturing step, or both, is carried out in a single vessel.

In some embodiments, the method further comprises a step of separating bacteria un-attached to said particle from bacteria attached to said particle at at-least one time point selected from the group consisting of: prior to, during, after the culturing step, and any combination thereof, thereby producing: (i) a composition comprising bacteria in planktonic form; and/or (ii) a composition comprising bacteria attached to said particle.

In some embodiments, the method further comprises the step of mixing composition (i) and composition (ii) at any desired ratio.

In some embodiments, the plurality of bacteria are provided within a vessel, and the method further comprises the steps of: adding at least one compound to said vessel; and determining any feature selected from: (i) bacterial diversity; (ii) bacterial relative abundance; (iii) bacterial load; (iv) any other effect of said at least one compound on said plurality of bacteria; (v) any change in said at least one compound, and (vi) any combination of (i) to (v); thereby evaluating the effect of: at least one compound on a plurality of bacteria, a plurality of bacteria on at least one compound, or both. In some embodiments, any change in the at least one compound comprises a chemical modification, a structural modification, or a combination thereof.

In some embodiments, the origin sample is selected from the group consisting of: derived from at least one origin, derived from at least one subject, is a microbiome sample, a skin sample, an oral sample, a fecal sample, a vaginal sample, comprises gut microbiota, and any combination thereof.

In some embodiments, the compound modifies: (i) the similarity level of at least 30% at the end of the culturing period, (ii) the bacterial load of at least 1·E⁴ per 1 gr particle at the end of the culturing step, or both (i) and (ii).

In some embodiments, modifies comprises: increasing or reducing any one of said similarity level, said bacterial load, or both.

In some embodiments, the method is carried out simultaneously in several single vessels.

In some embodiments, the bacterial diversity; the bacterial relative abundance; the bacterial load; and/or the other effect of the at least one compound on the plurality of bacteria is compared to the corresponding feature in the plurality of bacteria of the origin sample, and a modification in said feature is indicative that said at least one compound has an effect on the plurality of bacteria.

In some embodiments, the method further comprises culturing a control plurality of bacteria that is not exposed to the compound in a separate vessel, and wherein the bacterial diversity; the bacterial relative abundance; the bacterial load; and/or the other effect of the at least one compound on the plurality of bacteria is compared to the corresponding feature in the control plurality of bacteria, and wherein any modification in the feature is indicative that the compound has an effect on said plurality of bacteria.

In some embodiments, adding and/or determining is carried out using bacteria attached to said particle, bacteria un-attached to said particle, or both.

In some embodiments, the composition being a pharmaceutical composition for use in modulation of a microflora in a subject in need thereof.

In some embodiments, the composition is for use in an in-vitro method for evaluating the effect of: at least one compound on a plurality of bacteria, a plurality of bacteria on at least one compound, or both.

In some embodiments, the composition further comprises an acceptable carrier or excipient.

In some embodiments, the method further comprises a step of subjecting said bacteria to at least one compound; thereby generating a desired microbial profile, wherein said desired microbial profile comprises any one of: a predetermined bacterial α-diversity, a predetermined β-diversity, a predetermined bacterial relative abundance; a predetermined bacterial load, or any combination thereof. In some embodiments, subjecting said bacteria to at least one compound modifies, e.g., decreased or increased, the similarity level of at least 30% at the end of the culturing period.

In some embodiments, the co-culture and/or composition are enriched with bacteria from a different source.

In some embodiments, the method further comprises a step of harvesting the cultured plurality of bacteria. In some embodiments, the harvested cultured plurality of bacteria comprise a bacterial load of at least 1·E⁶ per 1 gr, e.g., per 1 gr particle. In some embodiments, a bacterial load of at least 1·E⁶ per 1 gr, e.g., per 1 gr particle, is obtained at the end of the culturing period. In some embodiments, the method further comprises a step of harvesting the co-cultured plurality of bacteria, and, optionally, any additional microorganism cultured and/or maintained in the culturing system, e.g., attached and/or un-attached to particles (referred to herein as “a biological biomass” or “biological cells”). In some embodiments, prior to harvesting the biological biomass is separated from the growth medium or the cell culture broth, e.g., by centrifugation, filtration and/or by letting the biological biomass to sediment within the culturing vessel.

In another aspect, there is provided an in-vitro method for evaluating the effect of: at least one compound on a plurality of bacteria, a plurality of bacteria on at least one compound, or both, the method comprising the steps of: providing a sample comprising a plurality of bacteria, contained within a vessel; contacting said plurality of bacteria with a particle, and allowing said plurality of bacteria to at least partially attach to said particle; culturing said plurality of bacteria at least partially attached to said particle in a growth medium for a period of less than 14 days, wherein said culturing comprises culturing under anaerobic conditions, thereby obtaining a co-culture comprising said plurality of bacteria; adding at least one compound to said vessel; and determining any one of: (i) bacterial diversity; (ii) bacterial relative abundance; (iii) bacterial load; (iv) any other effect of said at least one compound on said plurality of bacteria; (v) any change in the structure of said at least one compound, and (vi) any combination of (i) to (v); thereby evaluating the effect of: at least one compound on a plurality of bacteria, a plurality of bacteria on at least one compound, or both.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Further embodiments and the full scope of applicability of the present invention will become apparent from the detailed description given hereinafter. However, it should be understood that the detailed description and specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

FIG. 1 includes a non-metric multidimensional scaling (NMDS), β-diversity (Weighted Unifrac) plot showing the microbial diversity of a particle-attached bacteria fraction (marked as octagons), un-attached bacteria fraction (marked as squares), and of planktonic culture samples (marked as triangles), all produced simultaneously from the same origin sample (fecal sample; marked as plus). Several samplings were carried out from the particle-attached bacteria fraction, un-attached fraction, and from the planktonic culture. Each sampling is presented by one dot; therefore, several dots are shown for each system.

FIG. 2 includes a vertical bar graph showing Weighted Unifrac Similarity and bacterial count (qPCR measurements) of a composition to a reference sample at different time points (T1, T2 and T3) during culturing in a medium comprising a single-carbon source (glucose) compared to culturing in a multi-carbon source media (Glucose; Maltose; Trehalose; Starch). Origin fecal sample is considered as 100% (not presented on the graph).

FIG. 3 includes a vertical bar graph showing the effect of microelements supplementation to a glucose medium or to a multi-carbon medium on the similarity of a composition to a reference sample. The microelements used were: Manganese source; Copper source; and Iron source. Sampling was carried out at three time points (T1, T2 and T3) during culturing and the Weighted Unifrac similarity, and bacterial count (based on qPCR measurements) were evaluated.

FIG. 4 includes a graph showing the similarity level of a composition to an origin reference fecal sample (“Origin”). The results are presented as principal coordinates analysis (PCoA; PCA-based Metric Multidimensional scaling, MDS) plot. Samples comprising bacteria were cultured under aerobic and anaerobic conditions in different media (e.g., Brain-Heart Infusion Medium (BHI); or a pharmaceutical grade medium for growing human gut bacteria). An internal square surround the anaerobic conditions (anaerobic conditions are marked as AN).

FIG. 5 includes a vertical bar graph showing the similarity level and bacterial count (presented as a line plot; secondary Y axis; based on quantitative PCR (qPCR) measurements) of bacterial fecal samples cultured using different source media substrates under anaerobic conditions. Culturing was carried out in media according to an embodiment of the invention comprising: Glucose; Yeast extract; Peptone extract; Sodium Chloride; Disodium hydrogen phosphate in small—(PG2) or medium-scale production (Fermenter/Bioreactor PG2); or in small-scall culturing in BHI medium. Different time points were tested, reference fecal sample is marked as “Origin”.

FIG. 6 includes a vertical bar graph showing similarity and bacterial count level of a co-culture produced according to an embodiment of the invention using a multi-carbon source medium comprising: Glucose; Maltose; Trehalose; Starch; Yeast extract; Peptone extract; Sodium Chloride; Disodium hydrogen phosphate; Manganese source; Copper source; and Iron source. The results show the combined data from 3 processes using 3 origin samples from the same donor collected on different days. The samples were cultured independently in medium scale conditions (6 L).

FIGS. 7-9 include a non-metric multidimensional scaling (NMDS) of Bray-Curtis similarity metric performed at species level for the fungal, viral and bacterial communities, respectively, present in a composition produced according to an embodiment of the invention (marked as a square) and a reference stool sample (marked as a diamond). Fecal samples from healthy volunteers were also analyzed for their communities (marked as circles).

FIG. 10 includes a scatter plot that indicates the relative proportion/relative abundance of metabolic pathways tested in a composition produced according to an embodiment of the invention. X axis represents the origin sample; and Y axis represents the produced composition. Each point represents a metabolic super pathway, its location on either side of the gray dashed y=x line, means the pathway is enriched in one of the two compared samples.

FIGS. 11A-11B includes the growth preference of two bacterial families for a specific phase of the composition (attached to particles or un-attached). Each plot represents a different tested bacterium. Each dot on the graphs represents the delta between the relative abundance of the tested bacteria in the attached phase and its abundance in the un-attached phase in a sample obtained from a culture vessel at a specific time point. The skewness of the values towards a specific side of the graph represents the preference of the bacteria to a certain phase. A—shows a preference of a bacteria to the attached fraction; and B—shows a preference of a bacteria to the un-attached fraction.

FIGS. 12A-12B include plots, each representing a different β-diversity: A—Diversity recovery at Genus Level; and B—Diversity recovery at Species Level Every graph is composed of three panels that show comparison between two specific phases. First and second upper panels compare the combined phases vs. the un-attached and attached phases, respectively. Third lower panel compares the un-attached phase to the attached phase. Each dot on the graphs represents the delta between a subset of two chosen tested phases (attached, un-attached, combined) in a sample obtained from a culture vessel at a specific time point. Higher metric values of a specific phase compared to the other is observed by the skewness in the distribution from the zero value towards a specific side of the graph, whereas each side represents a different phase. Statistically significant differences between each two tested phases were determined using the Wilcoxon Non-Parametric Sign Rank test and are marked with an asterisk (*). The asterisk is shown on the side with significant enrichment.

FIG. 13 includes a heatmap showing the relative abundance of Akkermansia. The data is depicted by color, the darker the color, the higher the relative abundance of the bacteria within the tested sample or composition (starting from white—indicating no presence of bacteria; and up to 1: black, indicating maximal relative abundance). X Axis—represents specific sampling timepoints of bacterial fermentation; and axis Y—represents the origin sample used for culturing. Single origin Ori1, Ori 2 and Ori 3. Pooled origins: Ori1+Ori2+Ori3; or Ori2+Ori3.

FIG. 14 includes a flowchart demonstrating, as a non-limiting example, the steps of the herein disclosed method, in some embodiments thereof.

FIG. 15 includes a flowchart demonstrating, as a non-limiting example, the steps of the herein disclosed method, in some embodiments thereof.

FIG. 16 includes a flowchart demonstrating, as a non-limiting example, the steps of the herein disclosed method, in some embodiments thereof.

FIG. 17 includes a non-metric multidimensional scaling (NMDS), β-diversity (Bray-Curtis) plot showing the microbial diversity between 4 systems generated from 4 different fecal samples (derived from four different donors; marked as Donor 1-4); Donor #one provided samples at three different independent time points (marked as Experiment 1-3); i.e., several systems were generated simultaneously: 4 systems from 4 different donors; and additional 2 systems generated from Exp. 1 & 2 of Donor #1 (altogether 6 in-vitro culturing systems). Several samplings were carried out from the particle-attached bacteria phase. Each sampling is presented in the plot by one dot; therefore, several dots are shown for each system/donor.

FIG. 18 includes a box bar graph showing an average effect of Compound_A, as compared to an untreated control microbiome-based system, on the bacterial count (based on qPCR measurements) of 4 different systems generated from 4 fecal samples. Measurements were carried out at three different time points (TP1-TP3) during culturing. Note that the bacterial load results of all systems (4 treated and 4 untreated-control systems) are shown on the same graph. X-axis measures genome copies per μl and refers to bacterial count measurement by qPCR. This figure provides a non-limiting example for a method for evaluating the effect of a compound on a plurality of bacteria.

FIGS. 19A-19D include box bar graphs showing the effect of Compound_A on the α-diversity in 4 different systems generated from 4 different donors compared to an untreated control microbiome-based system. Measurements were carried out at 3 time points during the culturing period from the particle-attached bacteria phase. The α-diversity of a fecal sample before culturing is presented in each graph (“Origin”). This figure provides a non-limiting example for a method for evaluating the effect of a compound on a plurality of bacteria.

FIGS. 20A-20D include a non-metric multidimensional scaling (NMDS) plot showing the effect of Compound_A, as compared to control, on the β-diversity (Bray-Curtis) of a microbial population. Measurements were carried out at 3 time points during the culturing period from the particle-attached bacteria phase. Each plot shows the diversity in a different system, generated from a different donor at 3 subsequent time points during the culturing period (all time points are presented on the same plot). This figure provides a non-limiting example for a method for evaluating the effect of a compound on a plurality of bacteria.

FIG. 21 includes a heatmap showing different bacterial genera that were affected in view of an exposure of a plurality of bacteria, cultured in an in-vitro system, to Compound_A. Each experiment was carried out in 4 treated systems simultaneously (Samples 1-4). The darker the color, the bigger the change, patterned color means the change was a reduction in view of the exposure, and colors without pattern mean an increase in view of the exposure. Analysis was carried out as compared to an untreated control bacterial population. The intensity of the colors indicates the fold change level (see indicator level in the right rectangle); an average color indicator is also shown (Av.). A cross (x) indicates that the genus was not significant in the sample.

FIG. 22 includes a graph showing the effect of Compound_A and its derivate (Compound_A′) on the relative abundance of a bacterial population as compared to untreated bacterial population of a control system. The relative abundance is presented at the genus taxa level; and all systems were generated from the same fecal sample. Samples were taken at 5 time points. This figure provides a non-limiting example for a method for evaluating the effect of a compound on a plurality of bacteria.

FIG. 23 includes a graph showing the relative abundance of 9 selected genera in particle-attached and un-attached bacterial fractions following exposure to Compound_D. Upper panel shows treated fractions, lower panel shows the relative abundance of an un-treated system.

FIG. 24 includes a graph showing the relative abundance of 4 selected bacteria (marked as: “Bacterium 1-4”) in a generated control screening system and after addition of Compund_B. The relative abundance of each bacterium in the entire bacterial population was measured in both the particle-attached bacteria (“Attached”) and planktonic (“Plankton”) phases. The measurements were carried out at one time point for the control system (T1) and at two time points for the treated system (marked as: T1 and T2).

### DETAILED DESCRIPTION

### Methods of Preparations and Use

According to some embodiments, there is provided a method for producing a composition comprising a co-culture comprising a plurality of bacteria grown in the presence of a particle.

According to some embodiments, there is provided a method for producing a composition comprising a co-culture comprising a plurality of bacteria and a particle.

According to some embodiments, there is provided a method for obtaining a composition characterized by or having increased yields of gut bacteria.

According to some embodiments, there is provided an in-vitro system/model, e.g., an in-vitro gastrointestinal system, for evaluating the effect of at least one compound on a subject's bacterial population and/or a microbiome population; and/or the effect of a subject's bacterial population and/or a microbiome population on at least one compound.

According to some embodiments, there is provided an in-vitro system/model for the study and/or modulation of physiology, metabolic activity, prebiotic, probiotic or pathogenic characteristics of a subject's flora, optionally after an addition of at least one compound.

According to some embodiments, there is provided an in-vitro method for evaluating the mutual effect of at least one compound and a plurality of bacteria, e.g., a subject's bacterial population, and/or a microbiome population.

According to some embodiments, there is provided a method, and system for culturing a bacterial population (e.g., a plurality of gut bacteria).

According to some embodiments, there is provided a composition obtained by the method and/or system, as disclosed herein.

The methods, compositions and in-vitro systems/models according to the invention, in some embodiments, have several advantages, as demonstrated and detailed hereinbelow. In some embodiments, the method according to the invention comprises co-culturing of distinct bacteria in a single vessel, e.g., under the same culturing conditions. In some embodiments, the method according to the invention enables co-culturing of distinct bacteria using a medium comprising pharma-grade ingredients, e.g., components that are suitable for human consumption. In some embodiments, the method according to the invention enables production of complex microbial compositions and/or enables production of a composition comprising a co-culture comprising a plurality of bacteria grown in the presence of a particle, comprising at least 30% similarity to an origin sample, and having a bacterial load of at least 1·E⁴ per 1 gr particle, e.g., 1·E⁶ per 1 gr particle. In some embodiments, wherein the origin sample is a fecal sample, developing a controlled industrial process that would yield a composition that preserves the gut microbiome profile, e.g., of a healthy donor, could advantageously resolve manufacturing scalability, reproducibility and safety issues of carrying out fecal microbiota transplantation (FMT). In some embodiments, the in-vitro culturing system according to the invention, comprising a culturing step with a growth or culturing medium in the presence of particles as detailed herein, substantially maintains a high similarity (e.g., bacterial diversity and/or bacterial relative abundance) to a biological sample used to generate the system, and therefore can be utilized as an in-vitro model simulating a subject's bacterial population and/or a microbiome population. In some embodiments, the system according to the invention simulates or mimics a flora of a subject, e.g., the flora of a gastrointestinal tract, thus being utilized for determining bacterial population modulation or change once exposed, supplemented, or contacted with at least one compound/element. In some embodiments, the method and system according to the invention can be used as a high throughput screening assay and simultaneously compare the effect of different treatments/compounds (e.g., different bacteria, different drug derivatives, different prebiotics etc.) on a subject's bacterial population and/or microbiome population. In some embodiments, the method and system according to the invention enable to identify modulated bacteria (at any taxonomic level) that can be a therapeutic target for increasing the efficacy of a drug, e.g., by a drug-probiotic or a drug-prebiotic combination therapy aimed to restore/preserve the original or a healthier microbiome profile. In some embodiments, the method and system according to the invention enable to evaluate the effect of a subject's microbiome on at least one compound, e.g., any change in the chemical structure of the administered compound. In some embodiments, the method and system according to the invention can be used as part of a personalized medicine, wherein the effect of a compound of interest, e.g., a drug, is examined on a co-culture comprising a plurality of bacteria simulating a flora of a subject, e.g., by using the subject's microbiota or microbiome as an origin sample to generate the system. In some embodiments, using the system according to the method of the invention provides reproducible results and thus can accurately determine the effect of a compound on a plurality of bacteria that commonly colonize an environmental niche and/or plurality of bacteria that mimic relevant environmental conditions of a subject's microbiome, regardless of the origin of the biological sample (e.g., subject) used to establish the model. In some embodiments and without wishing to be bound to any theory, the system according to the invention, comprising bacteria in both planktonic and adhered form, better represents the natural behavior of the bacterial population in a subject's body, e.g., as compared to a system that includes only planktonic bacteria (cultured in the presence of a particle and/or cultured in the absence of a particle) or bacteria in adhered form. In some embodiments, the system is a stand-alone system, and is therefore not affected by the complexity of other physiological and/or metabolic processes existing in the human body.

In some embodiments, the phrase “bacteria grown in the presence of a particle” refers to bacteria that were in-vitro cultured in the presence of a particle. In some embodiments, “a composition comprising a co-culture comprising a plurality of bacteria grown in the presence of a particle” includes a composition comprising particle-attached bacteria, e.g., with substantially no planktonic bacteria that were cultured in the presence of a particle; a composition comprising planktonic bacteria that were cultured in the presence of a particle, e.g., with substantially no bacteria attached to particles; and a composition comprising both particle-attached bacteria and planktonic bacteria that were cultured in the presence of a particle. In some embodiments, the method can comprise a step of separating particle-attached bacteria from the particle. In some embodiments, the composition can be supplemented with bacteria that were cultured without a particle.

In some embodiments, the culturing comprises contacting the bacteria with a particle. In some embodiments, bacteria contacted with a particle is at least partially attached to the particle. In some embodiments, bacteria contacted with a particle is fully attached to the particle. In some embodiments, bacteria contacted with a particle is not attached to the particle. In some embodiments, bacteria contacted with a particle and is not attached to the particle comprises planktonic bacteria.

In some embodiments, the term “population(s)” with regards to the microorganisms, e.g., bacterial population or bacterial communities, is interchangeable with the term “communities” and refers to two or more groups, e.g., two or more bacterial or microbial groups, living symbiotically.

In some embodiments, the term “composition” includes the terms “in-vitro system(s) or model(s)”, “in-vitro culturing system(s)/model(s)”, in-vitro biological system model” and the like. In some embodiments, the at least one compound can be added at any step of producing the composition.

In some embodiments, the phrase “a system that better represents the natural behavior of the bacterial population in a subject's body” refers to a system comprising a bacterial population having at least 20%, at least 30%, at least 40%, at least 50%, at least 60%, at least 70%, at least 80%, at least 90%, at least 99%, or 100%, and any range therebetween, similarity to the bacterial population in the subject's body.

In some embodiments, the phrase “a system that better represents the natural behavior of the bacterial population in a subject's body” refers to a system comprising a bacterial population having at least 20%, at least 30%, at least 40%, at least 50%, at least 60%, at least 70%, at least 80%, at least 90%, at least 99%, or even 100%, and any range therebetween, similarity to the bacterial growth forms, e.g., planktonic and/or adhered state, in the subject's body. In some embodiments, the term “planktonic” comprises bacteria in non-adhered/attached state.

In some embodiments, the system or co-culture comprising the plurality of bacteria simulates a flora of a subject. In some embodiments, the phrase “simulates a flora/microflora of a subject” refers to a community of symbiotic bacteria and/or symbiotic microbes that mimic relevant environmental conditions of a subject's microbiome, e.g., exhibits similar in-vivo functions, physiology, metabolic activity and/or probiotic characteristic of a subject's microflora. In some embodiments, the system simulates different regions in the gastrointestinal tract, e.g., with respect to environmental condition such as pH level, physiological and/or chemical conditions, enzyme and their concentration etc.

As used herein, the terms “microbiome”, “microflora”, and “flora” are interchangeable and refer to the collection of microbes (including but not limited to bacteria, viruses, fungi such as yeast) found, constitute or known to reside in an environmental niche. In some embodiments, the term “microbiome” also includes the structural elements and/or metabolites/signal molecules of the microbes' community; and/or the surrounding environmental conditions in the environmental niche. In some embodiments, the plurality of bacteria are co-cultured with a collection of microbes found or known to reside in an environmental niche of a subject. Non-limiting examples of environmental niches include, but are not limited to, gut; skin; eye; bronchus; oral, e.g., saliva; upper respiratory tract; urogenital tract, and vaginal tissue, to name a few. In some embodiments, the term “microbiota” refers to the bacterial population, e.g., excluding non-bacterial populations.

In some embodiments, the plurality of bacteria used in accordance with the invention are being derived from an origin sample. As used herein, the term “being derived” is interchangeable with the term “originated” and “obtained” and refers to the source from which the plurality of bacteria for culturing/fermenting are obtained. In some embodiments, the origin sample comprises a collection of microbes (e.g., bacteria, viruses, fungi such as yeast) found, constitute or known to reside in an environmental niche. In some embodiments, the origin sample comprises bacteria and further comprises additional microorganisms, e.g., at least one taxonomic group of any one of: archaea, virus, fungi, or any combination thereof. In some embodiments, the plurality of bacteria are isolated from the source/origin, e.g., separated from other microorganisms or microbes present in the sample, prior to culturing. In some embodiments, the other microorganisms or microbes are cultured and/or maintained with the plurality of bacteria in the culturing system. In some embodiments, the term “virus” is or comprises bacteriophages.

In some embodiments, the term “culturing” includes the term “fermentation” and refers to the in-vitro maintenance, proliferation and/or growth of a microorganism, e.g., bacteria, in buffers and/or media of various kinds under laboratory or industrial conditions. A suitable culturing medium can be selected by the person skilled in the art and examples of such media include, but are not limited to, YCFA (Yeast Casitone Fatty Acids), BHI (Brain Heart Infusion), GAM (Gifu Anaerobic Medium), TSB (Tryptic Soy Broth), TYG (Tryptic Yeast Extract Glucose), FAB (Fastidious Anaerobic Broth) etc.

In some embodiments, the media comprises pharma-grade ingredients. In some embodiments, the media comprises food-grade ingredients. In some embodiments, the media comprises components suitable for veterinary use. The term “fermentation” has its ordinary meaning in the art. In some embodiments, the term “fermentation” is used herein to refer to a microbiological metabolic process comprising conversion of sugar(s) to acids and/or gases using, e.g., bacteria.

In some embodiments, the additional microorganisms or the collection of microbes present in the origin sample are co-cultured together with the plurality of bacteria. In some embodiments, other microorganisms or microbes are added separately or mixed into the culturing system. In some embodiments, any one of archaea, viruses and/or fungi are maintained in the co-culture, e.g., preserved and/or present, without any change in their quantity and/or in their relative abundance of taxa, vs. the origin sample.

In some embodiments, “preserved and/or present” refers to a population that is similar to the origin sample as defined herein. In some embodiments, the term “viruses” includes bacteriophages.

In some embodiments, the term “co-culturing” or “co-cultured”, as used herein, refers to the maintenance, proliferation and/or growth of the plurality of bacteria, and optionally, the one or more additional microorganisms or the microbes in the culturing system as described herein.

In some embodiments, the origin sample comprising the plurality of bacteria resembles a subject's microflora (such sample is referred to herein as “a resembled sample”), e.g., a healthy subject or a subject having a disease, disorder or condition such as dysbiosis. In some embodiments, “a resembled sample” is a sample comprising at least 30%, at least 40%, at least 50%, at least 60%, at least 70%, at least 80%, at least 90%, at least 99%, or even 100% identity or similarity to a subject's microflora and/or to a plurality of bacteria being derived/originated from a subject (e.g., as measured by α- or β-bacterial diversity; bacterial relative abundance; and/or bacterial load). In some embodiments, “a resembled sample” is created by mixing bacteria, e.g., single strains and/or a plurality of bacteria and/or co-cultured bacteria, based on a desired microbiome profile, e.g., in an environmental niche of a subject, such as in a gut, an eye, an oral cavity, a skin, a bronchus, a vagina, an upper respiratory tract, a urogenital tract, to name a few. In some embodiments, “a resembled sample” further comprises mixing of other microorganisms, e.g., archaea, viruses, fungi or any combination thereof.

In some embodiments, the origin sample comprises a pre-determined targeted or desired population of microorganisms, e.g., bacterial population. In some embodiments, the origin sample is used “as is” or following process as a starting material for the methods described herein. In some embodiments, processing of a sample comprises any one of: dilution, such as with a buffer, a culture media, or a combination; homogenization; partial or complete removal of non-floral matter, rough particulate matter, fibers or any combination thereof; and/or any other process known in the field of sample processing, e.g., fecal sample processing. In some embodiments, the composition is characterized by having at least 30% similarity to the pre-determined targeted population.

In some embodiments, the origin sample is a synthetic sample. In some embodiments, the origin sample is a biological sample. In some embodiments, the plurality of bacteria is derived from at least one origin. In some embodiments, the plurality of bacteria is derived from a combination of at least 2, at least 3, at least 4, at least 5 or more origins/sources. In some embodiments, the plurality of bacteria are sampled or derived from a donor (such as a human subject) having a desired microbiota population at the sampled environment. In some embodiments, the sample is derived from a healthy subject, a non-healthy subject and/or a subject having dysbiosis, e.g., in a certain environmental niche. In some embodiments, the sample is derived from more than one bacterial source. In some embodiments, the plurality of bacteria is derived from at least one environment. In some embodiments, the plurality of bacteria is derived from the sample. In some embodiments, the plurality of bacteria is at least partially derived from the sample. In some embodiments, the sample, composition and/or the co-culture are enriched with bacteria, and optionally other microorganisms, from a different source or origin.

In some embodiments, the term “synthetic sample” refers to a sample comprising a bacterial community that is created artificially by combining/mixing selected (two or more) bacteria species, e.g., that may commonly colonize a predetermined environmental niche. In some embodiments, the synthetic sample is or comprises selected groups of bacteria, at any taxonomic level, based on their relative abundance in a desired microbiota population in different subjects and/or different environmental niches.

In some embodiments, the sample used in the methods or systems according to the invention is or comprises any one of: a stored microbiota, a stored microbiome sample, plurality of bacterial population, bacterial colonies, particle-attached and/or planktonic bacteria or any combination thereof. In some embodiments, the sample is frozen or freeze dried prior to use in the methods or systems according to the invention. In some embodiments, the sample is stored at a temperature of lower than 8° C., e.g., at a temperature in the range of 2-8° C.

In some embodiments, the methods according to the invention comprises a step of providing a composition comprising a co-culture comprising the plurality of bacteria produced according to the invention, e.g., in frozen or freeze-dried form, e.g., for use in the evaluation method and/or as a starting material for producing the composition according to the invention.

In some embodiments, the plurality of bacteria is or comprises at least one bacterial population selected from: fecal bacterial population, gut bacterial population, eye bacterial population, oral cavity bacterial population (e.g., saliva bacterial population), skin bacterial population, bronchial bacterial population, vaginal bacterial population, upper respiratory tract bacterial population, urogenital tract bacterial population, or any combination thereof. In some embodiments, the plurality of bacteria is or comprises at least one bacterial population selected from: soil bacterial population, ground water bacterial population, open waters bacterial population, or any combination thereof. In some embodiments, the plurality of bacteria is or comprises gut microbiota.

In some embodiments, the origin sample comprising the plurality of bacteria is derived from a subject, e.g., from a fecal sample, an oral sample (e.g., a saliva sample), a skin sample, an eye sample, a bronchial sample, a vaginal sample, or any combination thereof. In some embodiments, the sample comprising the plurality of bacteria is derived from at least one, at least 2, at least 3, at least 4, at least 5 or more origins, e.g., the sample is a pool derived from several subjects.

In some embodiments, the flora, e.g., bacterial population, is separated from non-floral matter. In some embodiments, wherein the origin is a fecal sample, fecal flora are at least partially separated from non-floral matter of the fecal material, rough particulate matter and/or fibers. Separation can be carried out, e.g., by homogenization, centrifugation, filtration and/or by any other method known to the skilled in the art. In some embodiments, a fecal sample used according to the method of the invention comprises fibers originated from feces. In some embodiments, the bacterial population is at least partially separated from non-digested particles or matter.

In some embodiments, the sample comprises a soil sample.

In some embodiments, the sample is derived from plant(s).

As used herein, the term “plurality” refers to any integer equal to or greater than two. In some embodiments, the plurality of bacteria comprises at least two distinct bacteria. In another embodiment, the plurality of bacteria comprises at least 3, at least 4, at least 5, at least 6, at least 7, at least 8, at least 9, at least 10, at least 15, at least 20, at least 25, at least 30, at least 35, at least 40, at least 45, or at least 50 or more distinct bacteria. In some embodiments, the plurality of bacteria are from one or more taxonomic classification.

In some embodiments, the term “plurality of bacteria” refers to a bacterial population comprising at least two different strains or species of bacteria. In some embodiments, the bacterial population comprises from between two different types of bacteria, to up to a thousand different types of bacteria, or more.

In some embodiments, the term “distinct” refers to bacteria that are characterized as having differing growth, cultivation and/or proliferative conditions. In one embodiment, the phrase “bacteria that are characterized as having differing growth, cultivation and/or proliferative conditions” refers to bacteria that require distinct optimal conditions for growth, cultivation and/or proliferation. In some embodiment, the differing growth, cultivation and/or proliferation conditions are at least two optimal conditions wherein a first condition allows substantially optimal growth, cultivation and/or proliferation of a first bacterium, and a second condition allowing substantially optimal growth, cultivation and/or proliferation of a second bacterium.

In some embodiments, the differing growth, cultivation and/or proliferative conditions comprises any one of: metabolic requirements, nutritional requirements, pH, Temperature, aerobic, obligatory anaerobic, facultative anaerobic, microaerophilic, attached form, planktonic, growth medium, flow, shake, stir, agitation, static, moist, low-humidity, or any combination thereof. In some embodiment, the growth, cultivation and/or proliferation conditions comprise at least one obligatory anaerobic bacterium and at least one facultative anaerobic bacterium. In some embodiment, the differing growth, cultivation and/or proliferation conditions comprises at least two bacterial population selected from the group consisting of: obligatory aerobe, obligatory anaerobe, facultative anaerobe, microaerophile.

In some embodiments, the differing growth, cultivation and/or proliferative conditions are biological parameters selected from the group consisting of: metabolic requirements, nutritional requirements, growth medium, and any combination thereof.

In some embodiments, the differing growth, cultivation and/or proliferative conditions are metabolic requirements selected from the group consisting of: aerobic, obligatory anaerobic, facultative anaerobic, microaerophilic, and any combination thereof.

In some embodiments, the differing growth, cultivation and/or proliferative conditions are physical parameters selected from the group consisting of: temperature, moisture, low-humidity, flow, shake, stir, agitation, static, and any combination thereof.

In some embodiments, the differing growth, cultivation and/or proliferative conditions are chemical parameters selected from the group consisting of: pH, redox potential, gas composition, dissolved gases, and any combination thereof.

In some embodiments, the differing growth, cultivation and/or proliferative conditions are fermentation techniques selected from the group consisting of: Fed batch, Semi Batch, Batch, continuous, and any combination thereof.

In some embodiments, the differing growth, cultivation and/or proliferative conditions are bacterial growth form selected from the group consisting of: planktonic, attached form, and any combination thereof.

In some embodiments, the term “metabolic requirements” comprises any one of: carbon and energy sources, sources of basic elements (e.g., H, O, N), macroelements, microelements, vitamins, hormones, growth factors, CO₂ levels, oxygen levels, light, metabolic precursors or substrates, or any combination thereof.

In some embodiments, the compositions are produced by a method comprising: providing a plurality of bacteria being derived from an origin sample; contacting the plurality of bacteria with a particle, and allowing the plurality of bacteria to at least partially attach to the particle; and culturing the plurality of bacteria that are at least partially attached to the particle in a growth medium for a period of less than 14 days. In some embodiments, the culturing comprises culturing under anaerobic conditions. In some embodiments, the culturing comprises culturing under aerobic conditions.

In some embodiments, the compositions are produced by a method comprising: providing a plurality of bacteria being derived from an origin sample; culturing the plurality of bacteria in a growth medium for a period of less than 14 days, the culturing comprises culturing under anaerobic conditions.

In some embodiments, the compositions are produced by a method comprising: providing a plurality of bacteria being derived from an origin sample; culturing the plurality of bacteria in a growth medium for a period of less than 14 days, the culturing comprises culturing under aerobic conditions.

In some embodiments, the growth medium comprises a carbon source and a nitrogen source in a mole per mole/atom per atom ratio ranging from 50:1 to 1:50, or any value and range therebetween. Each possibility represents a separate embodiment of the invention. In some embodiments, carbon-to-nitrogen ratios, or ratios between embodied carbon types, either w/w or m/m, are based on dry form.

In some embodiments, the phrases “providing a plurality of bacteria being derived from an origin sample” and “providing a sample comprising a plurality of bacteria” are interchangeable.

In some embodiments, the plurality of bacteria or the sample comprising same are provided within a vessel, and the method further comprises the steps of: adding at least one compound to said vessel; and determining any one of: (i) bacterial diversity; (ii) bacterial relative abundance; (iii) bacterial load; (iv) any other effect of said at least one compound on said plurality of bacteria; (v) any change, e.g., in the structure of said at least one compound, and (vi) any combination of (i) to (v); thereby evaluating the effect of: at least one compound on a plurality of bacteria, a plurality of bacteria on at least one compound, or both.

In some embodiments, the in-vitro evaluation method and other uses according to the invention as defined herein can be carried out by using any one of the systems/compositions described herein, e.g., in the presence of particles, absence of particles, under culturing conditions comprising anaerobic conditions, under culturing conditions comprising aerobic conditions or any combination thereof. In some embodiments, the evaluation can be carried out in a system comprising particles, a system without particles and/or in both systems, e.g., as elaborated hereinabove and below. In some embodiments, the compound(s) can be added prior to and/or following addition of the particles into the culturing system, e.g., as elaborated hereinabove and below. The evaluation and/or determining step can be carried out in any of the bacterial fractions/phases produces according to the invention.

The phrase “contacting the plurality of bacteria with a particle” is used herein in its broadest sense and refers to any type of combining action which, e.g., brings the plurality of bacteria into proximity with the particle such that the bacteria can attach/adhere to the particles. In some embodiments, contact is carried out in a solution.

In some embodiments, contacting comprises combining the bacteria, the particles, and the solution in any order, any combination and/or sub-combination including any pre-mixing of two of the elements prior to adding the third element. For example, inoculating the bacteria in a solution, and thereafter culturing the bacteria in a solution comprising the particles. In some embodiments, the particle blend is prepared in a solution as a preceding step and is then combined with the plurality of bacteria.

In some embodiments, the contacting is carried out in a solution selected from: saline, phosphate buffered saline, a growth medium, or any combination thereof. In some embodiments, contacting comprises inoculating a liquid (e.g., a buffer, a culture media, or a combination thereof) containing particles with the plurality of bacteria; and incubating the particles with the plurality of bacteria for a time sufficient to allow the plurality of bacteria to at least partially attach to said particle. In some embodiments, contacting is or comprises culturing the bacteria in the presence of the particles. In some embodiments, contacting and culturing are carried out simultaneously or as subsequent steps.

In some embodiments, a time sufficient to allow the plurality of bacteria to at least partially attach or adhere to the particle is from 2 hours to 48 hours. In some embodiments, a time sufficient to allow the plurality of bacteria to at least partially attach/adhere to the particle ranges from: 2 hours to 12 hours, 2 hours to 24 hours, 6 hours to 24 hours, 10 hours to 24 hours, 15 hours to 20 hours, 20 hours to 40 hours, 6 hours to 40 hours, or any value and range therebetween. Each possibility represents a separate embodiment of the invention.

In some embodiments, the culturing comprises a fed-batch culture or a semi-fed-batch culture, e.g., some or all of the nutrients are provided to the culture during the cultivation process. In some embodiments, the fed-batch culture comprises a fixed volume fed-batch, a variable volume fed-batch or any combination thereof.

The term “fed-batch culture”, as used herein, refers to a method of culturing in which components, e.g., nutrients such as the carbon source and/or nitrogen source, are provided to the culture at at-least one time point subsequent to the beginning of the culturing/cultivation process. In some embodiments, the culturing is a batch culture, e.g., nutrients are provided at the beginning of the culturing process. In some embodiments, the culturing is a combination of fed-batch culture and batch culture. For example, one nutrient is provided at the beginning of the culturing and another nutrient is provided during the culturing.

In some embodiments, the growth medium comprises one type or more of nitrogen source. In some embodiments, the nitrogen source is or comprises at least one of: yeast extract, peptone, yeast peptone, enriched yeast peptone, casein, guar peptone, synthetic amino acid medium, wheat peptone, potato peptone, ammonium salts (variety of salts such as ammonium carbonate, ammonium chloride and ammonium nitrate) or any combination thereof.

In some embodiments, the culturing is or comprises culturing under anaerobic conditions.

In some embodiments, the term “anaerobic conditions” refer to conditions wherein free oxygen is lower than 500 ppm, 450 ppm, 400 ppm, 350 ppm, 300 ppm, 250 ppm, 200 ppm, 150 ppm, 100 ppm, 50 ppm, or 10 ppm, or any value and range therebetween. Each possibility represents a separate embodiment of the invention. In some embodiments, anaerobic conditions comprise conditions providing no free oxygen. In some embodiments, anaerobic conditions comprise conditions being devoid of free oxygen.

In some embodiments, the term “aerobic conditions” refers to conditions comprising the presence of molecular oxygen. In some embodiments, the oxygen concentration is above 20% (v/v out of the total gas present during culturing).

In some embodiments, the method for producing the composition according to the invention comprises a step of in-vitro culturing a complex bacterial population characterized as having differing growth, cultivation and/or proliferative conditions, thereby producing a composition comprising a co-culture comprising the complex bacterial population.

In some embodiments, the produced composition or co-culture comprises at least 30% similarity, at least 40% similarity, at least 50% similarity, at least 60% similarity, at least 70% similarity, at least 80% similarity, at least 90% similarity, at least 95% similarity, at least 99% similarity, or 100% similarity, to an origin sample, or any value and range therebetween. Each possibility represents a separate embodiment of the invention.

In some embodiments, the bacterial load of the produced composition or co-culture ranges between at least 1·E⁴ to at least 1·E⁹ per 1 gr particle. In some embodiments, the produced composition or co-culture comprises or is characterized by having a bacterial load of at least 1·E⁴ per 1 gr particle, at least 1·E⁵ per 1 gr particle at least 1·E⁶ per 1 gr particle, at least 1·E⁷ per 1 gr particle, at least 1·E⁸ per 1 gr particle, at least 1·E⁹ per 1 gr particle, at least 1·E¹⁰ per 1 gr particle, at least 1·E¹¹ per 1 gr particle, at least 1·E¹² per 1 gr particle, at least 1·E¹⁴ per 1 gr particle, at least 1·E¹⁶ per 1 gr particle, or at least 1·E²⁰ per 1 gr particle, or any value and range therebetween. Each possibility represents a separate embodiment of the invention.

In some embodiments, the culturing is carried out until said co-culture has a similarity greater than or equal to 30% to said origin sample, and a bacterial load in the range of 1·E⁴ per to 1·E⁹ per 1 gr particle.

In some embodiments a culturing period of less than 14 days comprises: 13 days at most, 12 days at most, 11 days at most, 10 days at most, 9 days at most, 8 days at most, 7 days at most, 6 days at most, 5 days at most, 4 days at most, 3 days at most, 2 days at most, 1 day at most, 5 hours at most, 3 hours at most, or any value and range therebetween. Each possibility represents a separate embodiment of the invention.

In some embodiments, a culturing period of less than 14 days comprises: 1 to 13 days, 2 to 13 days, 5 to 13 days, 2 to 10 days, 6 to 12 days, 4 to 11 days, 8 to 13 days, 2 to 9 days, 2 to 5 days, from 10 hours to 4 days, from 12 hours to 48 hours, from 5 hours to 6 days, from 6 hours to 5 days, from 6 hours to 14 days, from 3 hours to 6 days, from 3 hours to 5 days, from 12 hours to 24 hours, or any value and range therebetween. Each possibility represents a separate embodiment of the invention.

In some embodiments, the method comprises a culturing period ranging between 6 hours and 6 days, thereby producing a composition comprising a co-culture comprising a plurality of bacteria grown in the presence of a particle, the co-culture comprises: i) at least 50% similarity to an origin sample, and ii) a bacterial load of at least 1·E⁶ per 1 gr particle.

In some embodiments, the method comprises culturing in a growth medium comprising at least two types of carbon sources selected from: monosaccharide, di-saccharide, and polysaccharide, for a period ranging between 12 hours and 5 days, thereby producing a composition comprising a co-culture comprising a plurality of bacteria grown in the presence of a particle, the co-culture comprises: i) at least 70% Weighted Unifrac similarity to an origin sample, and ii) a bacterial load of at least 1·E⁸ per 1 gr particle.

In some embodiments, the culturing comprises culturing under any condition selected from: static, flow, stirring, shaking, agitating or any combination thereof. In some embodiments, the culturing comprises stirring the plurality of bacteria, e.g., at 50 to 750 revolutions per minute (RPM), 50 to 650 RPM, 100 to 750 RPM, 100 to 700 RPM, 150 to 700 RPM, 200 to 750 RPM, 130 to 690 RPM, 90 to 720 RPM, 70 to 550 RPM, 110 to 710 RPM, or any value and range therebetween. Each possibility represents a separate embodiment of the invention.

In some embodiments, the terms “flow”, “stirring”, “shaking” and “agitating” refer to conditions leading to movement or motion of the liquid phase within the culturing vessel. In some embodiments, the movement is axial, radial, mixed, distributed, or any combination thereof. The movement or motion can be carried out by using a mechanical mean, e.g., an impeller, a moving platform, rocker, shaker; manually; automatically or any combination thereof. In some embodiments, the term “static conditions” refers to conditions where no agitation or any other motion action (either manually, automatically and/or mechanically) is performed on the liquid phase.

In some embodiments, the culturing comprises subjecting the plurality of bacteria to a temperature in the range of: 32-39° C., 32-38° C., 33-38° C., 34-38° C., 35-38° C., 36-38° C., or 37-38° C., or any value and range therebetween. Each possibility represents a separate embodiment of the invention.

In some embodiments, the culturing comprises subjecting the plurality of bacteria to pH in the range of: 3.0-9.0, or in the range of 4.0-8.0, e.g., 3.0, 4.0, 5.0, 6.0, 6.2. 6.3, 6.4, 6.5, 6.5, 6.6, 6.7, 6.8, 6.9, 7.0, 7.0, 7.1, 7.2, 8.0, 9.0, or any value and range therebetween. Each possibility represents a separate embodiment of the invention.

In some embodiments, the culturing comprises subjecting the plurality of bacteria to pH of: 6.2 to 7.2, 6.2 to 7.1, 6.2 to 7.0, 6.3 to 7.2, 6.4 to 7.2, 6.5 to 7.1, 6.6 to 7.2, or 6.8 to 7.2, or any value and range therebetween. Each possibility represents a separate embodiment of the invention.

In some embodiments, prior to culturing the plurality of bacteria, the sample is diluted (e.g., with a buffer, a culture media, or a combination thereof) in a weight per volume (w/v) ratio ranging from: 1:1 to 1:300 such as, but not limited to, 1:5 to 1:60, 1:5 to 1:50, 1:10 to 1:25, 1:7 to 1:28, 1:9 to 1:30, 1:15 to 1:20, 1:6 to 1:24, 1:12 to 1:26, or 1:20 to 1:30, or any value and range therebetween. Each possibility represents a separate embodiment of the invention.

In some embodiments, the growth medium comprises one type of carbon source. In some embodiments, the medium comprises at least two types of carbon sources. In some embodiments, the medium comprises carbon sources from at least two groups selected from: monosaccharide, di-saccharide, and polysaccharide. In some embodiments, the growth medium further comprises at least one monosaccharide, at least one disaccharide, and at least one polysaccharide.

In some embodiments, the at least one monosaccharide, the at least one disaccharide, and the at least one polysaccharide are present in the growth medium in a weight per weight per weight (w/w/w) ratio of: 1:0:0, 1:1:0, 0:1:1, 0:1:0, 0:0:1, 1:0:1, 1:1:1 or any value and range therebetween. Each possibility represents a separate embodiment of the invention.

In some embodiments, the at least one monosaccharide, the at least one disaccharide, and the at least one polysaccharide are present in the growth medium in a weight per weight per weight (w/w/w) ratio of X:Y:Z, wherein any one of: X and Y; X and Z; and/or Y and Z are in a range from 0 to 10, or any value and range therebetween. Each possibility represents a separate embodiment of the invention.

In some embodiments, the monosaccharide is selected from: glucose (dextrose), fructose (levulose), galactose, or any combination thereof.

In some embodiments, the disaccharide is selected from: sucrose, lactose, maltose, trehalose, cellobiose, chitobiose, isomaltose, nigerose, maltulose, mannobiose, xylobiose, or any combination thereof.

In some embodiments, the term “polysaccharide” encompasses any polymer of carbohydrates composed of monosaccharide units that are linked to one another via a glycosidic bond. In some embodiments, the polysaccharide is selected from: alginate, starch, cellulose, pectin, arabinoxylans, glycogen, galactogen, inulin, or any combination thereof. In some embodiments, the polysaccharide comprises a synthetic polysaccharide. In some embodiments, a synthetic polysaccharide encompasses any non-naturally occurring polysaccharide.

In some embodiments, the growth medium further comprises a microelement being selected from: an iron source, a zinc source, a copper source, a manganese source, a selenium, an iodine, fluorine source, a molybdenum source, a cobalt source, a chromium source, a nickel source, their soluble salts, or any combination thereof.

In some embodiments, the method further comprises a step of substantially separating bacteria un-attached to said particle from bacteria attached to said particle, e.g., by filtration, washing and/or vortex, thereby producing: (i) a composition comprising bacteria in planktonic form; and/or (ii) a composition comprising bacteria attached to the particle. In some embodiments, the method further comprises a step of substantially removing bacteria un-attached/unadhered to particle(s) from the culturing system and/or from the growth medium, e.g., by filtration, washing and/or vortex. In some embodiments, un-attached/unadhered bacteria are or comprise planktonic bacteria. In some embodiments, un-attached bacteria are devoid of sessile bacteria. In some embodiments, the separation or removal is carried out at least once at a time point selected from: prior to and/or during the culturing step. In some embodiments, the separation or removal is carried out at the end of the culturing step.

In some embodiments, the term “substantially separating”, as used herein, refers to having less than 50%, e.g., less than 40%, less than 30%, less than 20%, lower than 10%, less than 5%, less than 4%, less than 3%, less than 2%, less than 1% or less, or any value and range therebetween, of un-attached bacteria in a composition comprising particle-attached bacteria and/or having less than 50%, e.g., less than 40%, less than 30%, less than 20%, lower than 10%, less than 5%, less than 4%, less than 3%, less than 2%, less than 1%, or less, or any value and range therebetween, of particle-attached bacteria in a composition comprising un-attached bacteria.

In some embodiments, the phrase “substantially removing bacteria un-attached/unadhered to particle(s)” refers to removal of at least 4%, at least 5%, at least 10%, at least 20%, at least 30%, at least 40%, at least 50%, at least 60%, at least 70%, at least 80%, at least 85%, at least 90%, at least 95%, at least 99%, or 100% of the un-attached/unadhered bacteria present in the co-culture, or any value and range therebetween. Each possibility represents a separate embodiment of the invention.

In some embodiments, the evaluation method is carried out in a system according to the invention using bacteria in planktonic form. In some embodiments, the evaluation method is initially carried out in a system comprising bacteria in planktonic form, e.g., prior to the contacting step and/or prior to attachment of the plurality of bacteria to the particles. In some embodiments, following the at least partial attachment, e.g., during the culturing step, the method is carried out in a system comprising any one of: particle-attached bacteria, bacteria in planktonic form, or both.

In some embodiments, the method further comprises a step of separating the plurality of bacteria cultured in the presence of a particle into different culturing systems to produce: i) a culturing system comprising bacteria in planktonic form that were initially cultured in the presence of a particle; and ii) a culturing system comprising bacteria attached to the particle. In some such embodiments, the compound can be added to any one of: the culturing system prior to the separation step; system (i), system (ii), or any combination thereof. The evaluation can be carried out separately in each system wherein the compound was added to.

In some embodiments, following the at least partial attachment, the culture comprising the plurality of bacteria can be divided into several vessels, e.g., for the evaluation of different compound(s)/condition(s). In some embodiments, the plurality of bacteria are cultured under the same culturing conditions.

In some embodiments, the contacting step, the culturing step, or both, is carried out within a vessel. In some embodiments, the contacting step, the culturing step, or both, is carried out within a single/one vessel, interconnected vessels, or communicating vessels.

In some embodiments, a vessel comprises any compartment compatible or configured to culturing and/or maintaining bacterial growth, proliferation, activity, and/or viability.

In some embodiments, the term “vessel” refers to any receptacle wherein bacteria can be cultured in conventional fermentation techniques, e.g., bioreactor(s), flask(s), test tube(s), microtiter dish(es), well-plate(s), multi-well plate assembly, petri-plate(s), and the like.

In some embodiments, the contacting step, the culturing step and/or the adding steps are carried out in a sequential manner in a single/one vessel.

In some embodiments, the vessel comprises an interconnected vessel, or communicating vessels. In some embodiments, the terms “interconnected vessels” and “communicating vessels” refer to a plurality of compartments or containers comprising a homogeneous liquid being connected sufficiently below the upper surface of the liquid.

In some embodiments, the in-vitro model simulates different regions in the gastrointestinal (GI) tract. In some such embodiments, several single vessels, each comprising a stand-alone system, can be positioned in a sequential manner, and interconnected, e.g., by a tube or a pipe, alternatingly above the upper surface of the liquid and below the lower surface of the liquid. Each vessel can represent the environmental conditions in the gastrointestinal tract. Simulation of the GI motility and/or functionality can be carried out, e.g., by utilizing mechanical means. In some embodiments, a liquid and/or gas is transferred through the interconnecting tube or pipe.

In some embodiments, the plurality of bacteria are not being divided into different vessels during culturing. In some embodiments, the method is being performed in multiple vessels. In some embodiments, the plurality of bacteria are being divided into different vessels prior to and/or during culturing, e.g., for simultaneously evaluating the effect of a compound on the plurality of bacteria. In sone embodiments, the plurality of bacteria are cultured under the same culturing conditions in the different vessels. In some embodiments, the culturing conditions are selected from the group consisting of: biological parameters (e.g., metabolic requirements, nutritional requirements, growth medium); metabolic requirements (e.g., aerobic, obligatory anaerobic, facultative anaerobic, microaerophilic); physical parameters (e.g., temperature, moisture, low-humidity, flow, shake, stir, agitation, static); chemical parameters (e.g., pH, redox potential, gas composition, dissolved gases); fermentation techniques (e.g., Fed batch, Semi Batch, Batch, continuous); bacterial growth form (e.g., planktonic, attached form), and any combination thereof.

In some embodiments, the evaluation method comprises the steps of: (a) providing a fecal sample of a subject; optionally, diluting the fecal sample, e.g., in a ratio ranging from 1:5 (g/ml) to 1:200 (g/ml), homogenizing and filtering the diluted sample; (b) inoculating particles with the fecal sample of step (a), e.g., in a ratio ranging from 1:2 (g/ml) to 1:10 (g/ml), and incubating or culturing the particles inoculated with the fecal sample for a period of less than 14 days, thereby producing a co-culture comprising a plurality of bacteria; (c) contacting the bacterial population with the at least one compound of interest; and (d) determining any one of: (i) bacterial diversity; (ii) bacterial relative abundance; (iii) bacterial load; (iv) effect of the at least one compound on the plurality of bacteria; (v) any change in the at least one compound, e.g., a chemical and/or a structural change, and (vi) any combination of (i) to (v). In some embodiments, diluting is in a buffer, a culture media, or a combination thereof.

In some embodiments, a pooled sample derived or obtained from more than one origin is used as part of the analysis method or system so as to determine whether a compound of interest is suitable for treatment in a subject in need thereof. As a non-limiting example, the effect of a compound of interest can be tested using at least 2 bacterial populations representing different sources, e.g., gut and skin, and determine whether a compound of interest is suitable for treatment of a disease or disorder related to a first microbial population as long as it does not modify a second microbial population. The following is an exemplary analysis according to which a compound identified not to modify a healthy gut microbial population or to rectify an altered gut microbial population, but also to modify a healthy skin microbial population, is determined as being unsuitable for treatment of a disease or disorder related to the gut. Alternatively, a compound identified to rectify an altered gut microbial population, and not to modify a healthy skin microbial population, is determined as being suitable for treatment of a disease or disorder related to the gut.

In some embodiments, as part of the analysis method or culturing system, different origin samples (e.g., from different donors, from the same donor but from different niches, a pool of origin samples mixed in different ratios or any combination thereof) are cultured in different vessels under the conditions of the instant application, e.g., to compare the effect of a compound on different origin samples.

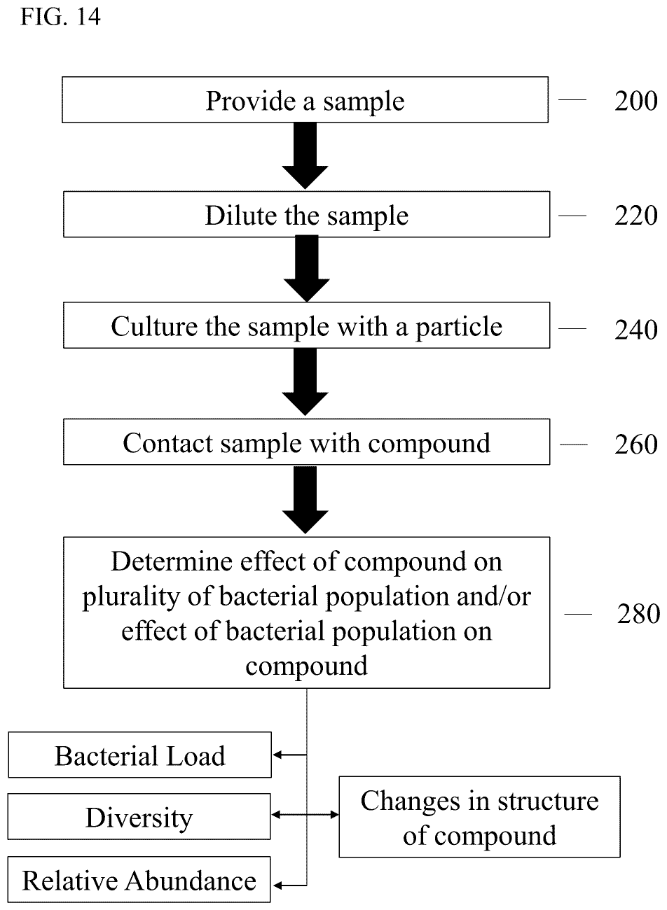

Reference is made to FIG. 14 describing as a non-limiting example of a set-up of the method of the invention. First, a sample comprising a plurality of bacteria (e.g., a microbial population comprising sample originated from at least one subject or origin population) is provided (step 200); subsequently, the sample can be diluted (step 220). The sample may be diluted in any solution such as PBS and/or any suitable medium which still maintains the bacteria natural surroundings and is suitable for growth. Dilution may be in any range which is suitable for bacterial preservation and/or growth, such as 1 gr sample:1 mL solution-1 gr sample-10 L solution, 1 gr sample: 10 mL solution-1 gr sample-5 L solution, or 1 gr sample:10 mL solution-1 gr sample-1 L solution. The sample or diluted sample is then cultured with a particle or a plurality of particles, under conditions allowing the bacteria to at least partially attach to the particle or the plurality of particles (step 240). The bacterial population at least partially attached to the particle or plurality of particles is subsequently contacted with a compound of interest, for a time and under conditions sufficient for the compound of interest to exert its activity over the bacteria (step 260). Subsequently, the effect of the compound of interest over the bacteria is determined (step 280). The output of the effect of the compound of interest over the bacteria, as determined in step 280, may be presented as bacterial load, bacterial diversity, bacterial relative abundance, bacterial abundance, and/or others, e.g., bacterial viability, particle-attached or adhered bacteria to planktonic ratio, gene expression profile, toxin production, among others. In addition, or alternatively, the effect of the bacterial population on the compound can be examined, by determining any changes in the concentration (e.g., due to consumption and/or degradation of the compound) and/or structure of the compound of interest, e.g., by metabolomics, analytical chemistry, biochemical methods, genomics, or by any other suitable method known in the art.

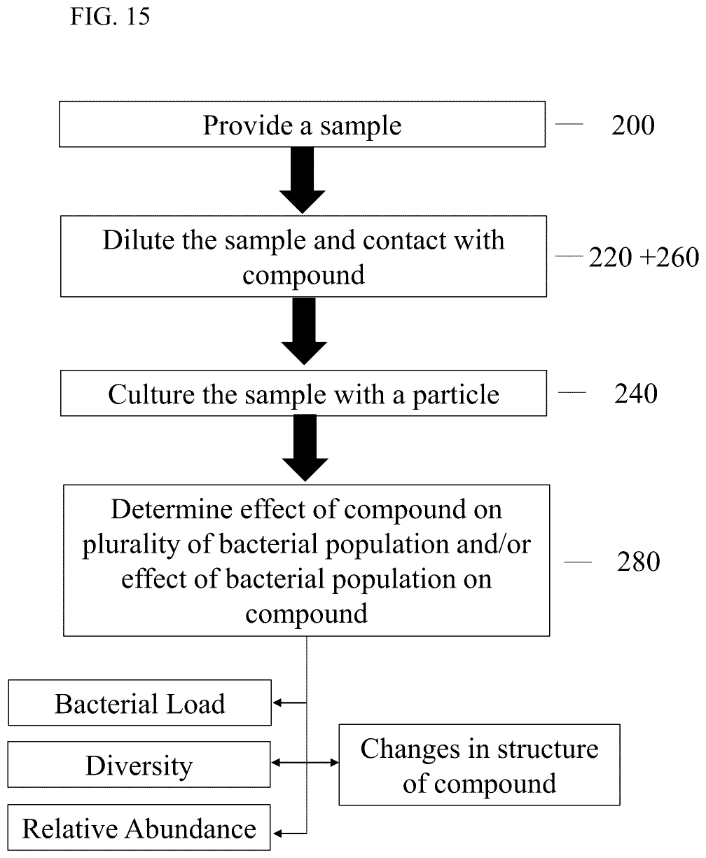

Reference is made to FIG. 15 describing as a non-limiting example of a set-up of the method of the invention. First, an origin sample comprising a plurality of bacteria (e.g., a microbial population comprising sample originated from at least one subject or origin population) is provided (step 200); subsequently, the sample can be diluted and is contacted with a molecule of interest (step 220+260). The sample may be diluted in any solution that is suitable for bacterial preservation and/or growth, as disclosed herein, and subjected to time and conditions sufficient for the compound of interest to exert its activity over the bacterial population. The sample or diluted sample is then cultured with a particle or a plurality of particles, under conditions allowing for the bacteria to at least partially attach to the particle or the plurality of particles (step 240). Subsequently, the effect of the compound of interest over the bacteria is determined (step 280). The output of the effect of the compound of interest over the bacteria, as determined in step 280, may be presented as bacterial load, bacterial diversity, bacterial relative abundance, bacterial abundance, and/or others, e.g., bacterial viability, particle-attached or adhered bacteria to planktonic ratio, gene expression profile, toxin production, among others. In addition, or alternatively, the effect of the bacterial population on the compound can be examined, by determining any changes in the concentration and/or structure of the compound of interest, e.g., by metabolomics, analytical chemistry, biochemical methods, genomics, or by any other suitable method known in the art.

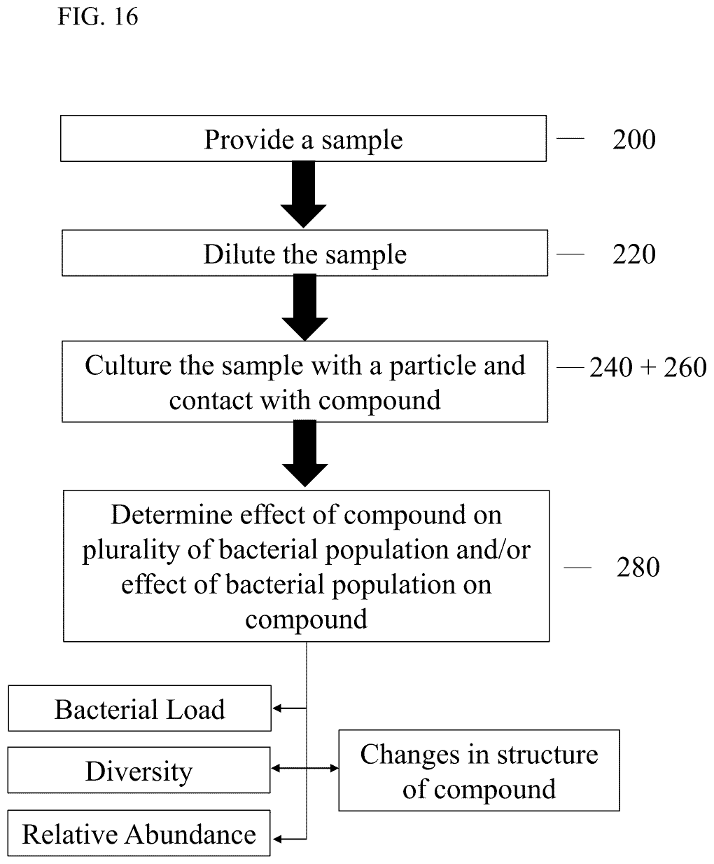

Reference is made to FIG. 16 describing as a non-limiting example of a set-up of the method of the invention. First, a sample comprising a plurality of bacteria (e.g., a microbial population comprising sample originated from at least one subject or origin population) is provided (step 200); subsequently, the sample can be diluted (step 220). The sample may be diluted in any solution that is suitable for preservation and/or growth, as disclosed herein. The sample or the diluted sample is then cultured with a particle or a plurality of particles, under conditions allowing for the bacteria to at least partially attach to the particle(s), and substantially simultaneously contacted with a compound of interest. The bacteria and the compound can be contacted for a time and under conditions sufficient for the compound of interest to exert its activity over the bacterial population (step 240+260). Subsequently, the effect of the compound of interest over the bacteria is determined (step 280). The output of the effect of the compound of interest over the bacteria, as determined in step 280, may be presented as bacterial load, bacterial diversity, bacterial relative abundance, bacterial abundance, and/or others, e.g., bacterial viability, particle-attached or adhered bacteria to planktonic ratio, gene expression profile, toxin production, among others. In addition, or alternatively, the effect of the bacterial population on the compound can be examined, by determining any changes in the concentration and/or structure of the compound of interest, e.g., by metabolomics, analytical chemistry, biochemical methods, genomics, or by any other suitable method known in the art.

In some embodiments, adding a compound at an early step in the method, e.g., prior to the bacteria being at least partially attached to the particle, advantageously enables determining the effect of the compound on an original bacterial profile, e.g., an original subject's bacterial profile. In some embodiments, an early stage comprises adding the compound prior to introducing the particles into the culturing vessel, e.g., contacting the compound with the origin sample, during inoculation, or any combination thereof. In some embodiments, allowing the bacteria to initially attach to the particles, prior to adding a compound, advantageously enables the bacteria to recover and form a more resilient/less susceptible bacterial population prior to the exposure to the compound.

In some embodiments, the method according to the invention comprises contacting the plurality of bacteria with at least one compound of interest. In some embodiments, the method according to the invention comprises contacting the plurality of bacteria with at least 2, at least 3, at least 4, at least 5 or more compounds of interest, either simultaneously or sequentially.

In some embodiments, the term “at least one compound” includes any element, condition and/or treatment or a number or list of elements, conditions and/or treatments. In some embodiments, the at least one compound is or comprises: a small molecule, a drug, a chemical substance, a peptide, a polypeptide, a protein, a carbohydrate, prebiotics, a bacteriophage, a bacterium, a fungus, a physical parameter, or any combination thereof. In some embodiments, a condition includes any culturing and/or growth conditions such as different growth media, different carbon sources, different nitrogen sources and the like.

In some embodiments, the term “a physical parameter” refers to any measurable physical characteristic or a number or list of measurable physical characteristics including, but not limited to, temperature; pH; stress factors; flow; shake; stir; humidity; gas, e.g., O₂, CO₂, N₂, and the like.

In some embodiments, the at least one compound is or comprises a microorganism being absent from the origin sample. In some embodiments, the at least one compound comprises a drug. In some embodiments, the drug is a drug approved for subject use or consumption.

In some embodiments, the method comprises contacting the plurality of bacteria with a drug. In some embodiments, the plurality of bacteria is contacted with a drug at a dose approved for subject consumption. In some embodiments, the plurality of bacteria is contacted with a drug in an effective amount approved for subject consumption, such as, but not limited to, to elicit the drug's therapeutic effect. In some embodiments, the dose used is lower or higher than the approved dose. In some embodiments, any derivative or salt of an approved drug is used. In some embodiments, the approved drug comprises any derivative, analog, or salt thereof, or any combination thereof.

In some embodiments, “an approved dose” is a clinically approved dose. In some embodiments, clinically approved is clinically approved for a human subject consumption or use.

Types of clinically approved drugs, as well as their dosing, are common and would be apparent to one of ordinary skill in the art.

In some embodiments, the subject is a eukaryote. In some embodiments, the subject is a plant. In some embodiments, the subject is an animal. In some embodiments, the subject is a mammal. In some embodiments, the subject is human.

The phrase “adding at least one compound to the vessel” or “subjecting the bacteria to at least one compound” is used herein in its broadest sense and refers, for example, to any type of combining action which exposes the plurality of bacteria to the compound. The plurality of bacteria can be exposed to, subjected to, or contacted with the compound at any step of the method.

In some embodiments, the at least one compound is added to the vessel prior to, substantially with, or shortly after addition of the sample or diluted sample to the vessel. The particles can be added prior to, substantially with or after addition of the compound. In some embodiments, the at least one compound is added to the vessel after the addition of the sample, and substantially with or shortly thereafter addition of the particles. In some embodiments, the bacterial population is exposed to the at least one compound during the culturing step. In some embodiments, the bacterial population is exposed to the at least one compound during contact of the plurality of bacteria with the particle(s), as a preceding step, during the culturing step, or at any combination thereof.

In some embodiments, the plurality of bacteria is exposed to the at least one compound only prior to the attachment of the plurality of bacteria to the particles; for the entire culturing period or both. In some embodiments, the compound can be contacted with the sample comprising the plurality of bacteria, with a diluted sample, with the co-culture comprising the plurality of bacteria, with particle-attached bacteria, with bacteria in planktonic form, e.g., grown in the presence of a particle, or any combination thereof.

In some embodiments, the evaluation method comprises a step of inoculating particles with the sample (or diluted sample), and contacting the particles inoculated with the sample with the at least one compound of interest.

In some embodiments, the step of determining or evaluating at least one of: (i) bacterial diversity; (ii) bacterial relative abundance; (iii) bacterial load; (iv) any other effect of the at least one compound on the plurality of bacteria; or (v) any change in the at least one compound, e.g., a chemical and/or structural change, is carried out at any time point throughout the steps of the method, e.g., during culturing. In some embodiments, determining is carried out prior to and/or following contact of the plurality of bacteria with the particles. In some embodiments, the step of determining at least one of: (i) bacterial diversity; (ii) bacterial relative abundance; (iii) bacterial load; (iv) any other effect of the at least one compound on the plurality of bacteria; or (v) any change in the at least one compound, is carried out at any time point during culturing, e.g., an hour, 5, 10, 15, 20 hours of culturing; at day 1, 2, 3, 4, 5, 6, 7, 8, 9, 10, 11, 12, 13 and/or 14 of culturing.

In some embodiments, the method further comprises transferring the plurality of bacteria to a different vessel at least once at a time point selected from: (i) during the contacting step; (ii) during the culturing period; (iii) prior to the step of adding the at least one compound, (iv) following the step of adding the at least one compound, or (v) any combination of (i) to (iv).

In some embodiments, the step of determining is carried out using the co-culture comprising the plurality of bacteria, the particle-attached plurality of bacteria, bacteria in planktonic form or any combination thereof. In some embodiments, the effect of the compound is compared between any one of: the co-culture comprising the plurality of bacteria, the particle-attached plurality of bacteria, bacteria in planktonic form.

In some embodiments, the bacterial diversity; the bacterial relative abundance; the bacterial load; and/or any other effect of the at least one compound on the plurality of bacteria, of the treated bacteria is compared to the corresponding feature in the plurality of bacteria in the origin sample or any cultured sample, and a change/modification in any feature is indicative that the at least one compound has an effect on the plurality of bacteria.

In some embodiments, any other effect of the at least one compound on the plurality of bacteria comprises: particle-attached to planktonic ratio, abundance of specific bacterium/bacteria, gene expression profile, protein and/or metabolite production, toxin production, suitability of a compound to generate a desired/predetermined microbial profile, suitability of a compound to enhance attachment of a specific bacterium to a particle and/or to enhance planktonic growth form of a specific bacterium within a plurality of bacterial population; or a combination thereof.

In some embodiments, the methods according to the invention are carried out simultaneously in several vessels. In some embodiments, several vessels may be used simultaneously in a high-throughput assay. In some embodiments, the method further comprises culturing “a control plurality of bacteria” that is not exposed to the compound in a separate vessel. In some embodiments, such high-throughput assay is carried out in systems generated from the same origin sample, e.g., so as to determine an optimal treatment or efficacy on a microbial population. In some embodiments, the bacterial diversity; the bacterial relative abundance; the bacterial load, any other effect of the at least one compound and/or any change in the at least one compound in the different treatments are compared to each other and/or to the corresponding feature in the control plurality of bacteria. In some embodiments, any change or modification is indicative that the at least one compound has an effect on the plurality of bacteria. In some embodiments, the effect is determined at several time points during and/or after exposure of the plurality of bacteria to the compound. In some embodiments, to determine the effect of the compound on the plurality of bacteria, a comparison between the different features is carried out at the same time point.

In some embodiments, the term “any change or modification” comprises: an increase, a decrease, a shift, a modulation, an alteration and/or a deviation in the microbial profile and/or bacterial load as compared to a reference sample, e.g., an origin sample, an untreated control plurality of bacteria, or any combination thereof.

In some embodiments, at the end of the culturing period, the cultured control system comprises at least 30% similarity, at least 40% similarity, at least 50% similarity, at least 60% similarity, at least 70% similarity, at least 75% similarity, at least 80% similarity, at least 85% similarity, at least 90% similarity, at least 95% similarity, at least 99% similarity, or 100% similarity to the origin sample used to generate the system, or any value and range therebetween. Each possibility represents a separate embodiment of the invention. In some embodiments, the produced control co-culture comprising the plurality of bacteria comprises at least 30% similarity, at least 40% similarity, at least 50% similarity, between 50-85% similarity, 60-90% similarity, 70-85% similarity, 75-99% similarity, 80-94% similarity, 85-100% similarity, 90-97% similarity, or 95-100% similarity to the provided origin sample, or any value and range therebetween. Each possibility represents a separate embodiment of the invention.

In some embodiments, the effect of the compound on the plurality of bacteria is examined by comparing the similarity level, the bacterial load and/or any other effect between a treated plurality of bacteria to any one of: (i) a control/cultured, e.g., untreated, plurality of bacteria; (ii) an origin provided sample, (iii) different treated plurality of bacteria, (iv) a co-culture comprising a plurality of bacteria, or any combination thereof. The effect can be examined by comparing different bacterial forms or fractions/phases, e.g., particle-attached bacteria, un-attached bacteria or a combination thereof.

In some embodiments, the bacterial load of an untreated control system (i.e., a generated system that was not exposed to compound(s) or element(s)) ranges between at least 1·E⁴ to at least 1·E⁹ per 1 gr particle, or any value and range therebetween, at the end of the culturing period. In some embodiments, an untreated control system comprises or is characterized by having a bacterial load of: at least 1·E⁴ per 1 gr particle, at least 1·E⁵ per 1 gr particle, at least 1·E⁶ per 1 gr particle, at least 1·E⁷ per 1 gr particle, at least 1·E⁸ per 1 gr particle, at least 1·E⁹ per 1 gr particle, at least 1·E¹⁰ per 1 gr particle, at least 1·E¹¹ per 1 gr particle, at least 1·E¹² per 1 gr particle, at least 1·E¹⁴ per 1 gr particle, at least 1·E¹⁶ per 1 gr particle, or at least 1·E²⁰ per 1 gr particle, or any value and range therebetween, at the end of the culturing period. Each possibility represents a separate embodiment of the invention.

In some embodiments, adding; contacting or exposing the plurality of bacteria to the at least one compound is under conditions suitable for the at least one compound to exert its effect on the bacterial population.

In some embodiments, a suitable condition is selected from: pH level, moist, low humidity, or any combination thereof. In some embodiments, a suitable condition comprises subjecting the bacteria to any one of: flow, shake, stir, agitation, static conditions or any combination thereof.

The compositions, systems and methods according to the invention, in some embodiments, can be used, inter-alia, to analyze metabolites produced by a subject's microbiota; explore how a specific microbial profile population is or can be modulated/modified in view of an exposure to different compound(s)/treatment(s); how different compounds are affected by a subject's microbial population; identify compounds that induce microbial profile modulation/modification, e.g., a desired/targeted microbial profile; identify effective combination treatments that can exert a synergistic or an additive effect, e.g., drug-probiotic, probiotic-prebiotic, drug-prebiotic therapies; as a platform for producing a composition having different desired microbial profiles, e.g., by adding to the culturing system different compounds and/or subjecting the plurality of bacteria to different conditions; for evaluating the suitability of a compound/condition to generate a desired microbial profile; as a platform for producing a composition enriched with bacteria, e.g., a specific bacterium attached to a particle, e.g., by adding to the system a compound/subjecting the plurality of bacteria to different conditions that promotes or enhances growth of bacteria that adhere to particles and/or bacterial attachment to the particles; for evaluating the suitability of a compound/condition to enhance a specific bacterial growth from of a particular bacterium within a plurality of bacterial population, e.g., adhered form; to identify bacterial strains that favor adherence to particles, strains that favor planktonic form, and strains that can grow in both forms, e.g., under specific conditions. In some embodiments, the condition in the system can simulate different regions in the gastrointestinal tract, to identify bacteria that favor a specific growth form in a subject.

In some embodiments, a favorable growth form for a specific bacterium is determined by separating bacteria un-attached to particle(s) from particle-attached bacteria, e.g., by filtration and/or by letting the particles sediment, and determining the abundance of the bacteria in each fraction, e.g., based on next generation sequencing (NGS) technology and/or whole genome sequence (WGS). In some embodiments, a bacterium is defined as favoring a specific growth form when its abundance is higher in one of the fractions.

In some embodiments, the term “microbial profile” comprises a bacterial diversity (e.g., α- or β-diversity); bacterial relative abundance; and/or bacterial load.

In some embodiments, the phrase “generate a desired or different microbial profile” refers to forming a plurality of bacteria having a designed/predetermined bacterial diversity (e.g., α- or β-diversity); bacterial relative abundance; and/or bacterial load, e.g., suitable for treating a disease, disorder or condition.

In some embodiments, the terms “modified”/“modifying” or “modulate”/“modulating” comprise increasing or reducing the similarity level of a microbiome profile, e.g., as compared to an origin or other reference microbial profile.

In some embodiments, the term “modifies” comprises rectifying the bacterial load, α-diversity, relative abundance, and/or β-diversity, of an un-healthy microbiota so as to resemble a healthy subject by at least 30%, at least 40%, at least 50%, at least 60%, at least 70%, by at least 80%, by at least 90%, by at least 99%, by 100%, or any value and range therebetween. Each possibility represents a separate embodiment of the invention.

In some embodiments, “modifies” comprises diverting the bacterial load, α-diversity, relative abundance, and/or β-diversity of a “non-responder patient” so as to resemble a “responder patient” by at least 30%, at least 40%, at least 50%, at least 60%, at least 70%, by at least 80%, by at least 90%, by at least 99%, by 100%, or any value and range therebetween. Each possibility represents a separate embodiment of the invention. In some embodiments, a “responder patient” comprises to a subject that achieved a response, e.g., a subject who is under remission and/or a subject who does no longer suffer from a disease, disorder or condition following treatment. In some embodiment, “a non-responder patient” comprises a subject for whom the disease, disorder or condition does not show reduction or improvement after the treatment.

In some embodiments, “reducing” or “reduce” is by at least 5%, at least 10%, at least 25%, at least 50%, at least 75%, or 100%, or any value and range therebetween. Each possibility represents a separate embodiment of the invention.

In some embodiments, “reducing” or “reduce” is by 5-50%, 25-75%, or 10-100%, or any value and range therebetween. Each possibility represents a separate embodiment of the invention.

In some embodiments, “increasing” or “increase” is by at least 5%, at least 10%, at least 25%, at least 50%, at least 75%, at least 100%, at least 250%, at least 500%, at least 750%, or at least 1,000%, or any value and range therebetween. Each possibility represents a separate embodiment of the invention. In some embodiments, “increasing” or “increase” is by 5-50%, 25-75%, 10-100%, 50-350%, 100-400%, 150-550%, 450-785%, or 200-1,000%, or any value and range therebetween. Each possibility represents a separate embodiment of the invention.

In some embodiments, the phrase “a composition enriched with bacteria or a bacterium attached to a particle” refers to a composition comprising a greater ratio of particle-attached bacteria to planktonic bacteria as compared to a control. In some embodiments, a control comprises a composition being cultured under essentially the same conditions without being subjected to the compound that promotes or enhances growth of bacteria that adhere to particles and/or bacterial attachment to the particles. In some embodiments, the ratio is at least 1.1-fold greater, at least 1.5-fold greater, at least 2-fold greater, at least 2.5-fold greater, at least 3-fold greater, at least 3.5-fold greater, at least 4-fold greater, at least 4.5-fold greater, at least 5-fold greater, at least 5.5-fold greater, at least 6-fold greater, at least 6.5-fold greater, at least 7-fold greater, at least 7.5-fold greater, or at least 8-fold, or any value and range therebetween. Each possibility represents a separate embodiment of the invention.

According to some embodiments, there is provided a composition produced according to the method of the invention. In some embodiments, the composition comprises: particle-attached bacteria and/or bacteria in planktonic form, e.g., bacteria cultured/grown in the presence of a particle. In some embodiments, the composition comprises the co-culture comprising the plurality of bacteria. In some embodiments, the composition comprises bacteria attached or adhered to a particle. In some embodiments, the composition comprises bacteria in planktonic form that was cultured/grown in the presence of a particle. In some embodiments, the compositions and/or the harvested co-culture produced according to the invention are enriched or supplemented with un-cultured bacteria, e.g., derived from a biological sample or a bacterial collection or repository, e.g., ATCC, DSMZ, and/or bacteria that were cultured in the absence of a particle. In some embodiments, the composition further comprises additional microorganisms, e.g., at least one of archaea, viruses, fungi or any combination thereof. In some embodiments, the composition comprises at least one bacterial species in a form partially attached to said particle and at least one bacterial species of planktonic bacteria.

In some embodiments, the composition is a synthetic composition. In some embodiments, the term “synthetic composition” refers to a composition comprising in-vitro grown or cultured bacteria. In some embodiments, a synthetic composition comprises an artificial composition. In some embodiments, a synthetic composition is a man-made composition, such as, but not limited to, a composition made or produced in a laboratory and/or a manufacturing site or facility. In some embodiments, a synthetic composition does not include a composition isolated or obtained from nature per se. In some embodiments, the composition is frozen, spray-dried or freeze-dried. In some embodiments, the composition is in the form of a dried powder. In some embodiments, the composition comprises: cryoprotectant, lyoprotectant, an anti-oxidant, or any combination thereof. In some embodiments, the composition comprises at least one metabolite that is produced in-vitro by at least one bacterium.

In some embodiments, the composition comprises bacteria in an aggregated form. In some embodiments, the terms “bacteria in aggregated form” and “bacterial clumps” are interchangeable and refer to the collection of individual bacterial particles into a single body.

In some embodiments, the composition comprises bacteria in the form of biofilm. In some embodiments, the biofilm is in the form of dried biofilm, e.g., in solid form, e.g., powder form. In some embodiments, the term “biofilm” refers to a community of bacteria embedded within a matrix comprising self-produced exopolysaccharides, e.g., that adheres to a particle's surface. In some embodiments, planktonic bacteria are stuck, entrapped, merged, or embedded, under, onto, or within the biofilm.

In some embodiments, the co-culture comprising the plurality of bacteria comprises particle-attached bacteria. In some embodiments, the co-culture comprises at least one bacterium in planktonic form. In some embodiments, the co-culture comprises or is a mixture of particle-attached bacteria and bacteria in planktonic form.

In some embodiments, the methods according to the invention comprises a step of removing bacteria un-attached to said particle or a step of separating bacteria un-attached to said particle from bacteria attached to said particle, thereby producing: (i) a composition comprising or enriched with bacteria attached to particles and substantially no planktonic bacteria that were cultured in the presence of a particle; and/or (ii) a composition comprising or enriched with planktonic bacteria that were cultured in the presence of a particle and substantially no bacteria attached to particles.

In some embodiments, the method comprises a step of mixing compositions (i) and (ii) in any desired proportion, e.g., to produce a final composition enriched with a specific bacterium that favors a specific growth form, e.g., planktonic or attached form, and as such is present in a higher abundance in such separated composition.

In some embodiments, “a composition comprising or enriched with bacteria attached to particles and substantially no planktonic bacteria that were cultured in the presence of a particle” refers to a composition comprising bacteria being in an attached growth form (e.g., more than 51%, such as at least 55%, at least 60%, at least 70%, or 100%, or any value and range therebetween) as compared to planktonic bacteria cultured in the presence of a particle.

In some embodiments, the phrase “a composition comprising or enriched with planktonic bacteria that were cultured in the presence of a particle and substantially no bacteria attached to particles” refers to a composition comprising planktonic bacteria that were cultured in the presence of a particle (e.g., more than 51%, such as at least 55%, at least 60%, at least 70%, or 100%, or any value and range therebetween) as compared to bacteria being in an attached growth form.

In some embodiments, the composition can comprise bacteria cultured in other growth forms, e.g., planktonic culturing.

In some embodiments, the composition according to the invention is administered to a subject immediately after culturing. In some embodiments, the composition is administered following storage, e.g., at room temperature, at a temperature in the range of 2-8° C. or a temperature below −18° C. In some embodiments, the composition is provided in a solid form, e.g., lyophilized, spray dried or frozen state. In some embodiments, the solid, e.g., lyophilized or spray dried, composition disclosed herein are stable at room temperature (e.g., at a temperature selected from the group consisting of: about 20, 21, 22, 23, 24, and 25° C., or any value and range therebetween) for a period of at least three months. In this context, the term “solid” refers to a physical state of a material.

In some embodiments, the composition is a solid composition.

In some embodiments, the composition is a spray-dried, or a freeze-dried composition.

Typically, the terms “lyophilization” and “freeze-dried” are interchangeable and refer to the process of freezing a solution followed by reducing the concentration of water, e.g., by sublimation to levels that do not support biological and/or chemical reactions. The resulting lyophilized composition can be stored for a long period of time while maintaining its stability. In some embodiments, the lyophilized composition can be used as a powder. In some embodiments, the powder or composition may be disposed in a suitable delivery vehicle or can be reconstituted by the addition of semi-liquid or a liquid solution. The volume added during the reconstitution can be similar, lower, or higher as compared to the initial volume of the solution before the lyophilization process.

In some embodiments, the composition comprises a carrier or an excipient. In some embodiments, the carrier is a veterinary, an agricultural and/or pharmaceutical acceptable carrier. The terms “carrier” and “excipient” are used herein interchangeably.

In some embodiments, there is provided a pharmaceutical composition comprising the composition disclosed herein and an acceptable carrier or excipient.

In some embodiments, the term “pharmaceutical composition” includes the term “a dietetic composition”, and “nutritional composition”.

In some embodiments, the terms “a dietetic composition”, “nutritional composition”, and nutraceutical composition” refer to a composition that is suited for being consumed as a food supplement, e.g., for supplementing the normal diet, correcting nutritional deficiencies, maintaining an adequate intake of certain nutrients, and/or for supporting specific physiological functions.

As used herein, the term “carrier”, “excipient” or “adjuvant” refers to any component of a pharmaceutical composition that is not the active agent. As used herein, the term “pharmaceutically acceptable” carriers, solvents, diluents, excipients, and vehicles generally refer to non-toxic, inert solid, semi-solid, liquid filler, diluent, encapsulating material, formulation auxiliary of any type, or simply a sterile aqueous solution, such as saline. In some embodiments, the term a “pharmaceutically acceptable carrier” refers to any diluent or a vehicle which is suitable for human or other animal use. Some examples of the materials that can serve as pharmaceutically acceptable carriers are sugars, such as lactose, glucose and sucrose, starches such as corn starch and potato starch, cellulose and its derivatives such as sodium carboxymethyl cellulose, ethyl cellulose and cellulose acetate; powdered tragacanth; malt, gelatin, talc; excipients such as cocoa butter and suppository waxes and hard fats; oils such as peanut oil, cottonseed oil, safflower oil, sesame oil, olive oil, corn oil and soybean oil; glycols, such as propylene glycol, polyols such as glycerin, sorbitol, mannitol and polyethylene glycol; esters such as ethyl oleate and ethyl laurate, agar; buffering agents such as magnesium hydroxide and aluminum hydroxide; alginic acid; pyrogen-free water; isotonic saline, Ringer's solution; ethyl alcohol and phosphate buffer solutions, as well as other non-toxic compatible substances used in pharmaceutical formulations. Some non-limiting examples of substances which can serve as a carrier herein include sugar, starch, cellulose and its derivatives, powered tragacanth, malt, gelatin, talc, stearic acid, magnesium stearate, calcium sulfate, vegetable oils, polyols, alginic acid, pyrogen-free water, isotonic saline, phosphate buffer solutions, cocoa butter (suppository base), emulsifier (e.g., carbomer, hydroxypropyl cellulose, sodium lauryl sulfate) as well as other non-toxic pharmaceutically compatible substances used in other pharmaceutical formulations. Wetting agents and lubricants such as sodium lauryl sulfate, as well as coloring agents, flavoring agents, excipients, stabilizers, antioxidants, and preservatives may also be present. Any non-toxic, inert, and effective carrier may be used to formulate the compositions contemplated herein. Suitable pharmaceutically acceptable carriers, excipients, and diluents in this regard are well known to those of skill in the art, such as those described in The Merck Index, Thirteenth Edition, Budavari et al., Eds., Merck & Co., Inc., Rahway, N.J. (2001); the CTFA (Cosmetic, Toiletry, and Fragrance Association) International Cosmetic Ingredient Dictionary and Handbook, Tenth Edition (2004); and the “Inactive Ingredient Guide”, U.S. Food and Drug Administration (FDA) Center for Drug Evaluation and Research (CDER) Office of Management, the contents of all of which are hereby incorporated by reference in their entirety. Examples of pharmaceutically acceptable excipients, carriers and diluents useful in the present compositions include distilled water, physiological saline, Ringer's solution, dextrose solution, Hank's solution, and DMSO. These additional inactive components, as well as effective formulations and administration procedures, are well known in the art and are described in standard textbooks, such as Goodman and Gillman's: The Pharmacological Bases of Therapeutics, 8th Ed., Gilman et al. Eds. Pergamon Press (1990); Remington's Pharmaceutical Sciences, 18th Ed., Mack Publishing Co., Easton, Pa. (1990); and Remington: The Science and Practice of Pharmacy, 21^{st} Ed., Lippincott Williams & Wilkins, Philadelphia, Pa., (2005), each of which is incorporated by reference herein in its entirety. The presently described composition may also be contained in artificially created structures such as liposomes, ISCOMS, slow-releasing particles, and other vehicles. Liposomes include emulsions, foams, micelles, insoluble monolayers, liquid crystals, phospholipid dispersions, lamellar layers and the like. Liposomes for use with the presently described peptides are formed from standard vesicle-forming lipids which generally include neutral and negatively charged phospholipids and a sterol, such as cholesterol. A variety of methods are available for preparing liposomes as reviewed, for example, by Coligan, J. E. et al, Current Protocols in Protein Science, 1999, John Wiley & Sons, Inc., New York, and see also U.S. Pat. Nos. 4,235,871, 4,501,728, 4,837,028, and 5,019,369.

The carrier may comprise, in total, from about 0.1% to about 99.99999% by weight of the pharmaceutical compositions presented herein.

In some embodiments, the composition is for pharmaceutical use. In some embodiments, the composition is for agricultural use. In some embodiments, the composition is for veterinary use.

In some embodiments, there is provided a method of preventing or treating a disease, disorder, or condition (e.g., dysbiosis) and/or for modulating a microflora in a subject in need thereof, the method comprises administering to the subject a therapeutically effective amount of the composition according to the invention.

The terms “a therapeutically effective amount” and “an effective amount” refer to the amount required to prevent, ameliorate and/or treat a disease, disorder, or condition. The effective dose may be changed depending on the gender, age and weight of the subject, the disease or condition and its severity and on any other factor which can be recognized by the skilled in the art.

In some embodiments, the pharmaceutical composition is for use in the treatment or prevention of dysbiosis in a subject in need thereof. In some embodiments, the composition as disclosed herein is for use in the preparation of a medicament for the treatment or prevention of dysbiosis in a subject in need thereof.

In some embodiments, the pharmaceutical composition is for use in modulating a microflora in a subject in need thereof, e.g., for treating or preventing C. diff infection, Ulcerative Colitis, or Atopic Dermatitis in a subject in need thereof.

In some embodiments, the term “modulating a microflora” includes reversing established bacteria which is typically associated with a disease, health condition or a clinical symptom and/or achieving colonization of a beneficial bacteria in any tissue and/or surface of any body part of an organism, including, but not limited to, any external or internal surface of the body and deep layers of the skin which are typically associated with a disease, health condition or a clinical symptom.

As used herein, the term “dysbiosis” is characterized by altered, imbalance, impairment and/or dysfunction of the microbiota and/or microbiome in any tissue and/or surface of any body part of an organism, including, but not limited to, any external or internal surface of the body and deep layers of the skin which is typically associated with a disease, health condition or a clinical symptom. The imbalance can be in any microbial community, including, without any limitations, a gastrointestinal tract, skin, oral, bronchial, vaginal, or rectal imbalance to name a few. In some embodiments, the dysbiosis is a tumor dysbiosis.

In some embodiments, the term “surface of any body part of an organism” refers to an external surface of the body that can be seen by unaided vision, e.g., the skin of the face, throat, scalp, chest, back, ear, neck, hand, elbow, knee, and other skin sites, and to a surface of an internal body part, e.g., which is a part of the internal anatomy of an individual, such as, but not limited to, the oral cavity, the gastrointestinal tract and the lower genital tract including, but not limited to, the vagina.

In some embodiments, dysbiosis comprises imbalance of microbial flora in a substrate or media. In some embodiments, the substrate or media comprises or is soil.

In some embodiments, an altered microbiota refers to a flora that has diverted from the homoeostatic microbiota, such as in a non-healthy subject. In some embodiments, an altered microbiota comprises different bacterial diversity, bacterial relative abundance, and/or bacterial load, compared to a healthy microbiota. As used herein, the term “healthy subject”, refers to a subject having a natural flora representing a healthy subject. In some embodiments, the altered microbiota comprises pathogenic microbes. In some embodiments, an altered microbiota is associated with a medical condition and/or is harmful to a subject's health, e.g., a human subject.

In some embodiments, the subject is a mammal. In some embodiments, the subject is an animal. In some embodiments, the subject is a human subject. The subject may be male or female.

A pharmaceutical composition may take any physical form necessary for proper administration. The composition can be administered in any suitable form, including but not limited to, a liquid form, a gel form, a semi-liquid (e.g., a liquid, such as a viscous liquid, comprising some solid) form, a semi-solid (a solid comprising some liquid) form, or a solid form. Compositions can be provided in, for example, a tablet form, a pessary form, a cream form, a suppository form, a capsule form, a liquid form, a food form, a chewable form, a non-chewable form, a transbuccal form, a sublingual form, a slow-release form, a non-slow-release form, a sustained release form, or a non-sustained-release form.

In some embodiments, the composition is formulated for administration by a mode selected from: peroral, rectal, parenteral, mucosal, vaginal, nasal, local, topical, pulmonary, ocular, oral, buccal administration, or any a combination thereof.

A pharmaceutically acceptable carrier suitable for the preparation of unit dosage form of a composition as described herein for peroral administration is well-known in the art.

In some embodiments, similarity comprises at least one characteristic selected from the group consisting of: functionality, e.g., the ability of bacteria to influence metabolic processes of a subject and/or ameliorate any disease, disorder or condition in a subject; potential metabolic routes/pathways, e.g., the potential to produce, synthesize, consume and/or utilize certain metabolites/organic compounds; relative abundance; α-diversity; recovered operational taxonomic unit (recovered OTU) or recovered amplicon sequence variant (ASV); β-diversity and any combination thereof.

In some embodiments, the term “similarity” includes resemblance between bacterial community or population present within the co-culture as compared to the bacterial community or population present within the origin sample. In some embodiments, the term “similarity” includes resemblance between other microorganisms, present within the composition, comprising at least one of: archaea; viruses, e.g., bacteriophage; fungi or any combination thereof. Similarity can be determined by any method known to the skilled person.

In some embodiments, similarity is determined by α-diversity metrics. In some embodiments, α-diversity metrics comprises any one of: Observed Species, Total Genera Found, Shannon, Chaol, Simpson or any combination thereof. In some embodiments, α-diversity is a measure that considers the diversity or richness of taxa, e.g., bacterial taxa, in a sample. In some embodiments, the α-diversity is compared between two communities (or samples). In some embodiments, when relating to α-diversity similarity metric, the diversity or richness of the taxa in the composition or co-culture is compared to the diversity or richness in the origin sample, e.g., at any taxonomic level. In some embodiments, Shannon, Chaol, and Simpson refers to diversity as described in: Kim B R, Shin J, Guevarra R, Lee J H, Kim D W, Seol K H, Lee J H, Kim H B, Isaacson R. Deciphering Diversity Indices for a Better Understanding of Microbial Communities. J Microbiol Biotechnol. 2017 Dec. 28; 27(12):2089-2093. doi: 10.4014/jmb.1709.09027. PMID: 29032640.

In some embodiments, similarity is determined by β-diversity metrics. In some embodiments, β-diversity metrics comprises any one of: Jaccard, Unweighted Unifrac similarity, Weighted Unifrac similarity, Generalized Unifrac similarity, Bray-Curtis similarity, or any combination thereof. In some embodiments, Generalized Unifrac Similarity is carried out as elaborated in Jun et. al. “Associating microbiome composition with environmental covariates using generalized UniFrac distances”. Bioinformatics. 2012 Aug. 15; 28(16): 2106-2113.

In some embodiments, the similarity is determined by Unweighted Unifrac Similarity. In some embodiments, the similarity is determined by Weighted Unifrac Similarity. In some embodiments, “Unweighted Unifrac Similarity” (“Unweighted Unifrac”) and “Weighted Unifrac Similarity” (“Weighted Unifrac”) refer to a similarity measure between two communities (or samples) that considers both: I. the presence/absence data of bacterial population; and II. the phylogenetic relatedness of bacteria. The Weighted Unifrac Similarity also considers the relative abundance of the bacterial population.

In some embodiments, the similarity is determined by Bray-Curtis. In some embodiments, “Bray-Curtis dissimilarity index” is a non-phylogenetic beta-diversity similarity measure between two communities (or samples) that considers the abundance of bacterial population within the sample. In other embodiments, using a non-phylogenetic beta-diversity metric may result with a lower similarity value vs. other similarity metrics that consider the phylogenetic relatedness of the bacteria (e.g., Weighted Unifrac Similarity, Unweighted Unifrac Similarity).

In some embodiments, the co-culture comprises at least 30% similarity to an origin sample when the similarity is determined by a metric that considers the phylogenetic relatedness of the bacteria using next generation sequencing (NGS) technology and/or whole genome sequencing (WGS). In some embodiments, a metric that considers the phylogenetic relatedness comprises any one of: Diversity recovery, Weighted Unifrac similarity, Unweighted Unifrac similarity, Generalized Unifrac similarity or any combination thereof. The analysis can be carried out at any taxonomic level. It is to be understood that various similarity metrics may be employed according to the invention, however, some methods may result with a lower similarity value, e.g., using a non-phylogenetic beta-diversity metric resulted according to an embodiment of the invention with a lower similarity value vs. other similarity metrics tested that consider the phylogenetic relatedness of the bacteria, e.g., lower than 30%.

In some embodiments, the similarity is determined by relative abundance. In some embodiments, the term “relative abundance” is the percent of a particular taxa, e.g., bacterial taxa, relative to the total abundance of taxa and is used herein to compare the distribution of a genus and/or any other bacterial taxonomic level among a community, e.g., a bacterial community, within a sample.

In some embodiments, the α- and/or β-diversity similarity are calculated from recovered OTU and/or recovered ASV. In some embodiments, the terms “recovered OTU”, “recovered ASV”, or “diversity recovery” refer to the percent of taxa observed in a tested sample out of the total taxa observed in another sample, e.g., fecal origin sample.

In some embodiments, the similarity is compared to a processed origin sample, e.g., homogenized and/or filtered.

Similarity indices can be based on next generation sequencing (NGS) technology, e.g., ribosomal 16S RNA or encoding DNA, and/or whole genome sequence (WGS), as would be apparent to one of ordinary skill in the art. Similarity can be determined at any taxonomic level, e.g., phylum, family, genus, species, and/or strain.

In some embodiment, the origin sample comprises a pre-determined bacterial population, and the co-culture is characterized as having at least 30% similarity to the pre-determined bacterial population. In some embodiments, the term “pre-determined” refers to a custom-made origin composition comprising plurality of bacterial population selected according to a specific need, e.g., suitable for treating a subject having gut dysbiosis. In some embodiments, the term “pre-determined” refers to bacterial population that commonly colonize an environmental niche. In some embodiments, the origin sample further comprises additional microorganisms (e.g., selected from archaea, viruses, fungi, or any combination thereof), and the co-culture is characterized as having at least 30% similarity to the additional microorganism populations present within the sample.

In some embodiments, the provided plurality of bacteria or the plurality of bacteria in the origin sample belong to at least 5 and up to 600 species of bacteria as determined by 16S NGS technology, e.g., at least 10, at least 15, at least 20, at least 25, at least 30, at least 35, at least 40, at least 45, at least 50, at least 55, at least 60, at least 65, at least 70, at least 75, at least 80, at least 85, at least 90, at least 100, at least 110, at least 120, at least 130, at least 140, at least 150, at least 160, at least 170, at least 180, at least 190, at least 200, or more species of bacteria, and/or belong to at least 2 bacterial genera and up to 250 genera of bacteria as determined by 16S NGS technology, e.g., at least 10, at least 15, at least 20, at least 25, at least 30, at least 35, at least 40, at least 45, at least 50, at least 55, at least 60, at least 65, at least 70, at least 75, at least 80, or more bacterial genera, or any value and range therebetween. Each possibility represents a separate embodiment of the invention.

In some embodiments, the provided plurality of bacteria or the plurality of bacteria in the origin sample belong to at least 35 and up to 80 observed genera and/or belong to at least 120 and up to 300 observed species, as determined by 16S NGS technology, or any value and range therebetween. Each possibility represents a separate embodiment of the invention.

In some embodiments, the composition or co-culture has a similarity of at least 30% with respect to the diversity or richness of the plurality of bacteria.

In some embodiments, the co-culture comprising the plurality of bacteria comprises at least 60%, at least 70%, at least 80%, at least 90%, at least 95%, at least 99% or 100% similarity to an origin sample, or any value and range therebetween. Each possibility represents a separate embodiment of the invention.

In some embodiments, bacterial load is determined by any method known to the skilled person such as, but not limited to, viable bacterial colony forming unit (CFU), quantitative polymerase chain reaction (qPCR), flow-cytometry, live-dead staining, Propidium Monoazide qPCR (PMA-qPCR), microscopy, a metabolic assay, spectrophotometry, or any combination thereof. Methods for determining bacterial load, such as disclosed herein, are common and would be apparent to a skilled artisan.

In some embodiments, the term “bacterial load” is interchangeable with the term “bacterial count”. In some embodiments, the co-culture or composition comprise viable bacteria and the bacterial count is measured by CFU.

In some embodiments, the bacterial load in the composition produced according to the invention is calculated per gr dry-form particle introduced into the culturing medium or vessel. In some embodiments, the bacterial load is calculated per gr dry-form particle used to contact with the plurality of bacteria. In some embodiments, when measuring the bacterial load of the co-culture in the composition, the weight of the particles added to the culturing system is considered. In some embodiments, when measuring the bacterial load of the co-culture in the composition, the weight of the particles in the final composition is considered.

In some embodiments, the term “a particle” comprises a plurality of one type or several types of particles and refers to a substance/material which is adapted, configured or suitable for at least one bacterium adherence/attachment and/or growth thereto. In some embodiments, the particles comprise a surface to which the bacteria can adhere to.

In some embodiments, in the context of the particles, the term “surface” is interchangeable with the term “surface area” and refers to the external surface of the particle. In some embodiments, the particles are porous. In such an embodiment, the term “surface” includes the outer surface of the porous structure of the particle. In some embodiments, the particles are non-porous. In some embodiments, the particles are a mix of porous and non-porous particles. In some embodiments, the term “porous” refers to the void (i.e., “empty”) spaces in a material. In some embodiments, the term “porous” includes having an un-even surface area.

As used herein the term “particle(s)” “nanoparticle(s)”, “microparticle(s)”, “nanosphere(s)”, and “microsphere(s)” are used interchangeably.

In some embodiments, the particle does not originate from the origin sample. In some embodiments wherein the origin is a fecal sample, the particle is not undigested fibers of feces. In some embodiments, the origin sample is devoid of a particle as disclosed herein.

In some embodiments, the particle used in accordance to the invention is a water-insoluble active agent. In some embodiments, the term “water-insoluble active agent” refers to a particle that has a solubility in water at 25° C. of less than 5 mg/ml, preferably below 1 mg/ml, and most preferably less than 0.1 mg/ml.

In some embodiments, the particle used in accordance to the invention is water-insoluble in a physiological pH. In some embodiments, the particle used in accordance to the invention is a water-insoluble in pH ranging from about 1.0 to 8.0.

In some embodiments, “a particle” comprise particles having a diameter in the range of 5 microns to 1 cm in diameter. In some embodiments, the particles range from of 1 micron to 50 millimeters. In some embodiments, the particles are at least 5, at least 10, at least 15, at least 20, at least 30, at least 40, at least 50, at least 60 microns, at least 70, at least 80, at least 90, at least 100, at least 200, at least 300, at least 400, at least 500, at least 600, at least 700, at least 800, at least 900, at least 1 cm in diameter, or any range or value therebetween. Each possibility represents a separate embodiment of the invention.

In some embodiments, the average diameter of the particles is in the range of 1 to 1,500 microns. In some embodiments, average diameter of the particles is in the range of 50 to 1,200 microns, 50 to 1,100 microns, 50 to 1,000 microns, 55 to 1,200 microns, 55 to 1,000 microns, 57 to 1,200 microns, or 60 to 1000 microns, including any range therebetween. Each possibility represents a separate embodiment of the invention.

In some embodiments, a diameter is an average diameter. In some embodiments, a diameter is a maximal diameter. In some embodiments, a diameter is a minimal diameter.

In some embodiments, the term “particles” includes any particle, e.g., in the embodied size range, in any shape. In some embodiments, the particle can be round, amorphous, irregular, sphere, elliptical, flower, cubic, spherical, elongated, rod-shaped, having any other shape, or any combination thereof.

In some embodiments, the particle is selected from: microcrystalline cellulose (MCC), dicalcium phosphate (DCP), seeds, a polysaccharide, or any combination thereof. In some embodiments, the particle comprises a plurality of one type of particle. In some embodiments, the particle comprises a plurality of types of particles.

In some embodiments, the particle comprises a combination of calcium and cellulose. In some embodiments, the particle comprises a combination of phosphate and cellulose. In some embodiments, the particle comprises a combination of calcium, phosphate, and cellulose. In some embodiments, the particle comprises or consists of MCC and DCP.

In some embodiments, the particles are completely immersed in the culture medium. In some embodiments, the particles are at least partially immersed in the culture medium.

In some embodiments, the particle comprises MCC and DCP in a weight per weight (w/w) ratio ranging from 5:1 (w/w) to 1:5 (w/w), 4:1 (w/w) to 1:4 (w/w), 3:1 (w/w) to 1:3 (w/w), 2:1 (w/w) to 1:2 (w/w), 1:1 (w/w), 5:1 (w/w) to 1:4 (w/w), 5:1 (w/w) to 1:3 (w/w), 5:1 (w/w) to 1:2 (w/w), 5:1 (w/w) to 1:1 (w/w), 4:1 (w/w) to 1:5 (w/w), 4:1 (w/w) to 1:1 (w/w), 3:1 (w/w) to 1:5 (w/w), 2:1 (w/w) to 1:5 (w/w), or 1:1 (w/w) to 1:5 (w/w), or any value and range therebetween. Each possibility represents a separate embodiment of the invention. In some embodiments, all w/w ratios herein relating to the particles, are based on anhydrous forms.

In some embodiments, a seed is selected from: cranberries, passionfruit, herbals, oat, or any combination thereof.

In some embodiments, the particle comprises or consists of a food grade particle. In some embodiments, food grade particle comprises or consists of a polysaccharide, a fat crystal, a protein, or any combination thereof. In some embodiments, a food grade particle comprising a fat crystal is selected from: glycerol monooleate, glyceryl stearyl citrate, or a combination thereof. In some embodiments, a food grade particle comprising polysaccharide is selected from: corn starch, starch nanocrystals, cellulose nanocrystals, microcrystalline cellulose, nano- or methyl cellulose, chitin, chitosan, or any combination thereof. In some embodiments, a food grade particle comprising a protein is selected from: β-lactoglobulin, lactoferrin, lactoferrin-polysaccharide, bovine serum albumin, gelatin, collagen, soy protein isolate, pea protein, Zein, or any combination thereof. In some embodiments, the food grade particle is selected from: flavonoid (tiliroside), wax, shellac-xanthan gum, or any combination thereof.

In some embodiments, the weight per weight ratio between the particles and the sample according to the method of the invention ranges between 1:2 to 1:10, 1:3 to 1:10, 1:4 to 1:10, 1:5 to 1:10, 1:6 to 1:10, 1:7 to 1:10, 1:8 to 1:10, or 1:9 to 1:10, or any value and range therebetween. Each possibility represents a separate embodiment of the invention.

In some embodiments, the solution, e.g., buffer, or medium used during contacting or culturing, and the particles are at a volume per weight (v/w) ratio ranging from 1:1 to 200:1, or any value and range therebetween. Each possibility represents a separate embodiment of the invention.

In some embodiments, the terms “attached” and “adhered” with regards to the bacteria comprises adsorption to a surface, e.g., via weak interactions and/or stronger interactions, e.g., by flagella, pili, lipopolysaccharides, exopolysaccharides, collagen-binding adhesive proteins, etc.

In some embodiments, “additional microorganisms are at least partially attached to the particles” comprises microorganisms other than bacteria, e.g., archaea, viruses, and/or fungi, attached to the particle, particle-attached bacteria and/or to a matrix formed by the attached bacteria, and refers to at least 0.01% attached-“additional microorganisms” out of the total “additional microorganisms” present in the culturing system or at the end of the contacting or culturing step.

In some embodiments, after attachment of the bacteria to the particle(s), some or all of the bacteria can be detached, and/or other bacteria in suspension can attach to the particle, particle-attached/adhered bacteria and/or to a matrix formed by the attached/adhered bacteria. In some embodiments, “bacteria at least partially attached to the particle” comprises bacteria attached to the particle-attached/adhered bacteria and/or to a matrix formed by the attached/adhered bacteria. In some embodiments, the phrase “plurality of bacteria at least partially attached to the particle” refers to at least 0.01%, to at least 0.05%, to at least 1%, at least 2%, at least 3%, at least 4%, at least 5%, at least 10%, at least 20%, at least 30%, at least 40%, at least 50%, at least 60%, at least 70%, at least 80%, at least 85%, at least 90%, at least 95%, at least 99%, or 100%, or any value and range therebetween, particle-attached/adhered bacteria out of the total bacteria present in the culturing system or at the end of the contacting or culturing step. The total bacteria can be determined by methods for determining bacterial load, such as disclosed hereinbelow. The attached and un-attached fractions can be evaluated by separating the two fractions, e.g., for evaluating the percentage of the attachment and/or for individually analyzing each fraction. In some embodiments, individual analysis comprises separating the two fractions and determining the bacterial count/load in each fraction. In some embodiments, separating the fractions or removal of un-attached bacteria is carried out by or comprises filtration and/or gravitational sedimentation, e.g., centrifugation. In some embodiments, the term “filtration” includes all separation techniques as well as any other processes utilizing a filter that can separate the fractions.

### General

All numeric values are herein assumed to be modified by the term “about”. The term “about” generally refers to a range of numbers that a person skilled in the art would consider equivalent to the recited value (e.g., having the same function or result). In many instances, when a numerical value is preceded by the term “about”, the term “about” is intended to indicate ±10%.

The terms “comprises”, “comprising”, “includes”, “including”, “having” and their conjugates mean “including but not limited to”. The term “consisting of” means “including and limited to”.

The term “consisting essentially of” means that the composition, method, or structure may include additional ingredients, steps and/or parts, but only if the additional ingredients, steps and/or parts do not materially alter the basic and novel characteristics of the claimed composition, method, or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an”, and “the”, include plural references unless the context clearly dictates otherwise. As used in this specification and the appended claims, the term “or” is generally employed in its sense including “and/or” unless the content clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical, microbiological and medical arts.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination or as suitable in any other described embodiment of the invention. Certain features described in the context of various embodiments are not to be considered essential features of those embodiments unless the embodiment is inoperative without those elements.

The descriptions of the various embodiments of the present invention have been presented for purposes of illustration but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

Various embodiments and aspects of the present invention as delineated hereinabove and as claimed in the claims section below find experimental support in the following examples.

Other terms as used herein are meant to be defined by their well-known meanings in the art.

Additional objects, advantages, and novel features of the present invention will become apparent to one ordinarily skilled in the art upon examination of the following examples, which are not intended to be limiting. Additionally, each of the various embodiments and aspects of the present invention as delineated hereinabove and as claimed in the claims section below finds experimental support in the following examples.

### EXAMPLES

Generally, the nomenclature used herein, and the laboratory procedures utilized in the present invention include molecular, biochemical, microbiological, and recombinant DNA techniques. Such techniques are thoroughly explained in the literature. See, for example, “Molecular Cloning: A laboratory Manual” Sambrook et al., (1989); “Current Protocols in Molecular Biology” Volumes I-III Ausubel, R. M., ed. (1994); Ausubel et al., “Current Protocols in Molecular Biology”, John Wiley and Sons, Baltimore, Maryland (1989); Perbal, “A Practical Guide to Molecular Cloning”, John Wiley & Sons, New York (1988); Watson et al., “Recombinant DNA”, Scientific American Books, New York; Birren et al. (eds.) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Culture of Animal Cells—A Manual of Basic Technique” by Freshney, Wiley-Liss, N. Y. (1994), Third Edition; “Current Protocols in Immunology” Volumes I-III Coligan J. E., ed. (1994); Stites et al. (eds), “Basic and Clinical Immunology” (8th Edition), Appleton & Lange, Norwalk, CT (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, CA (1990); Marshak et al., “Strategies for Protein Purification and Characterization—A Laboratory Course Manual” CSHL Press (1996); all of which are incorporated by reference. Other general references are provided throughout this document.

In the Examples below, culturing of plurality of bacteria derived from a fecal/stool or saliva sample (obtained from healthy human donors) was used, as an embodiment, to exemplify the capability of the method according to the invention to produce a composition comprising a co-culture having a high similarity to an origin sample, e.g., for pharmaceutical use and/or as an in-vitro screening system. To evaluate the effect of a medicament on a microbiome sampled from diseased subjects in the screening model, a fecal sample was obtained from diseased donors. In the Examples, “bacterial samples”, “stool samples” and “origin fecal sample” are all used to refer to the origin sample used to produce the composition.

An exemplary origin fecal sample was used in the experiments below comprised between 42-58 bacterial Genera (with an average of 50.1); and 131-196 Observed bacterial Species (with an average of 168.1). The data was obtained from five different experiments using five different origin samples and is presented as a range wherein the minimal and maximal values are the minimal and maximal value obtained in the five different experiments, in brackets is the average value of the five different experiments.

Typically, a sample derived from a human donor, e.g., a fecal sample, comprises various types of microorganisms, including bacteria, archaea, viruses, and fungi. Within each microorganism type, there are distinct populations/communities requiring different maintenance, growth, cultivation and/or proliferative conditions. As such, maintaining a high similarity of such various populations in-vitro is challenging, especially when cultured together in a single vessel.

Furthermore, each sample origin has a different microbial composition, further increasing the challenge of maintaining similarity, reproducibility, and process scaleup.

It was reported in the literature that most gut microorganisms are considered unculturable or difficult to culture as an eco-system [see, e.g., Lagier, J. C., Dubourg, G., Million, M., Cadoret, F., Bilen, M., Fenollar, F., Levasseur, A., Rolain, J. M., Fournier, P. E. and Raoult, D., 2018. Culturing the human microbiota and culturomics. Nature Reviews Microbiology, 16(9), pp. 540-550, Liu, Sijia, Christina D. Moon, Nan Zheng, Sharon Huws, Shengguo Zhao, and Jiaqi Wang. “Opportunities and challenges of using metagenomic data to bring uncultured microbes into cultivation.” Microbiome 10, no. 1 (2022): 1-14. Mabwi, H. A., Kim, E., Song, D. G., Yoon, H. S., Pan, C. H., Komba, E. V., Ko, G. and Cha, K. H., 2021. Synthetic gut microbiome: advances and challenges. Computational and structural biotechnology journal, 19, pp. 363-371].

The Examples below show that despite the complexity and variation among origin samples, the similarity of various communities to the origin sample, e.g., the bacterial population, as well as their functionality were preserved during culturing and scale up according to the exemplified embodiments.

### Material and Methods

### Similarity Parameters

In order to obtain the relative abundance of each taxa in the co-culture, similarity indices commonly used in the art were calculated based on 16S Next Generation Sequencing (NGS) or Whole-Genome Shotgun (WGS) technology.

“Relative abundance” is the percent of a particular taxa relative to the total abundance of taxa and is used to evaluate the distribution of a taxa among a community within a sample. In the Examples, the relative abundance was compared between two tested samples.

“α-diversity”, is a measure that considers the number of taxa in a sample. In the Examples, the α-diversity was compared between communities (or samples).

“Recovered OTUs” (and/or recovered ASVs and/or Diversity Recovery), is a similarity measure between two communities (or samples) that considers the percent of OTUs (and/or ASVs) observed in the processed sample (composition) out of the total OTUs (and/or ASVs) observed in the origin sample. “Recovered OTUs” (and/or ASVs) refers to bacterial diversity/richness within the processed and/or cultured sample (composition) as compared to the OTUs (and/or ASVs) in the origin sample.

“β-diversity” presented in “Unweighted Unifrac Similarity” (“Unweighted Unifrac”) or “Weighted Unifrac Similarity” (“Weighted Unifrac”) is a similarity measure between two communities (or samples) that considers both: I. the presence/absence data of taxa, e.g., bacterial population; and II. the phylogenetic relatedness of the taxa. The Weighted Unifrac Similarity also considers the relative abundance of the taxa in the population.

“β-diversity” presented in Bray-Curtis similarity metric, is a similarity measure between two communities (or samples) that refers to the abundance of taxa within the sample.

Each dot on the β-diversity plot represents a whole microbial/viral/fungal composition profile of one sample. Samples with similar microbial/viral/fungal profiles are presented closer to each other, while samples with different profiles are presented at a distance from each other.

“Chaol Richness Index”, “Shannon Index”, “Simpson Reciprocal” or any other parameter used in the examples for analysis of population dynamics has a meaning as known in the art.

### Bacterial Count

In the examples below, the bacterial load was calculated per gr dry-form particle introduced into the culturing vessel. The particles' weight was calculated according to the particles used to contact with the bacteria.

Determination of bacterial load was carried out using the following known methodology: a DNA based quantification (quantitative polymerase chain reaction—qPCR); and a colony forming unit (CFU) assay as known in the art. Results are presented in logarithmic scale per 1 gr dry-form particle introduces into the vessel.

CFU measurement. Serial dilutions of the samples were conducted, and bacteria were plated in triplicates onto CDC plates. The plates were incubated at 37° C. in anaerobic conditions for 48-72 h prior to CFU counting.

qPCR measurement. Absolute abundance quantification was carried out using quantitative real-time PCR with a standard curve that was devised with plasmid DNA containing a single copy of the 16S gene in 10-fold serial dilutions. The total DNA was calculated by normalizing to the standard curve and to the average of the assumed genome size.

In the examples below the term “bacterial count” and “bacterial load” are used interchangeably.

### Processing and Culturing Procedure of the Origin Biological Sample

Unless indicated otherwise, all processes were carried out under anaerobic conditions. Stool samples were collected inside a disposable stool container and transferred into anaerobic conditions. Preparation of an inoculum from a fecal sample was carried out under anaerobic conditions. When testing the effect of aerobic culturing, processing was carried out in either anaerobic or aerobic conditions.

One mg to 450 gr feces were mixed with sterile anaerobic saline solution (50 ml-6 L), blended until the mixture was homogeneous, and filtered. The filtrate (referred to herein as Fecal Liquid—FL/“origin” samples; considered as 100% in similarity calculations) comprising the bacterial population was transferred to the different fermentation vessels (50 ml-6 L). The bacterial population were cultured in a single vessel under anaerobic conditions in a medium as elaborated below in the presence of particles MCC:DCP (20:80 e.g., 20 gr MCC; 80 gr DCP). Contacting and culturing in the presence of particles was carried out for a period of less than 14 days.

For assessing the effect of a compound and a bacterial population, one mg to fifty gr feces were mixed with 1-150 ml sterile anaerobic saline solution. The bacterial samples were cultured in a single vessel under anaerobic conditions in a medium (1-150 ml) as elaborated herein below in the presence of 10-1,200 gr particles MCC:DCP (20:80 or 50:50; MCC:DCP). Culturing was carried out for a period of less than 14 days.

### Media Exchange and Treatment Sampling Process

The media was exchanged several times throughout the culturing procedure. Prior to media exchange, mixing/stirring was stopped to allow the particles to sediment. After media exchange, mixing was resumed.

For assessing the effect of a compound on a bacterial population or the effect of a bacterial population on a compound, culturing was carried out either under static conditions or under stirring conditions. Sampling was carried out at specific time points following the exposure of the bacterial population to the tested compound.

Culturing conditions included: Stirring in the range of 50-750 RPM; Temperature—32-39° C.; and pH—4.4-8.0.

Unless specified otherwise, for Similarity Parameters and Bacterial Load measurements, sampling was carried out from the particle-attached bacteria phase. Separation of the phases was carried out by letting the particles sediment, by centrifugation, washing or by filtration.

### Sampling Process

For measuring similarity and bacterial load, sampling was carried out during the culturing procedure at several timepoints (designated as: T1 or TP1, T2 or TP2, T3 or TP1).

### Media Composition

The media compositions used are elaborated in the Examples below. Pharma or food grade components were used.

### Example 1

### Culturing a Plurality of Bacteria in One-Vessel in the Presence of a Particle

In the following example, the effect of culturing a plurality of bacteria in the presence of a particle on the similarity level was assessed.

For this purpose, a fecal sample was processed and cultured in the presence of particles as elaborated above under the Material and Methods section. As a comparison, a processed fecal sample was cultured under the same conditions in the absence of particles. The similarity was determined using the Weighted Unifrac Similarity metric at the end of the culturing. The similarity of a composition produces according to both culturing conditions were compared to a reference fecal sample. Measurements were carried out at the end of the culturing period from: (i) the planktonic culturing; and (ii) from both the planktonic and particle-attached bacteria phases of a culturing carried out in the presence of a particle.

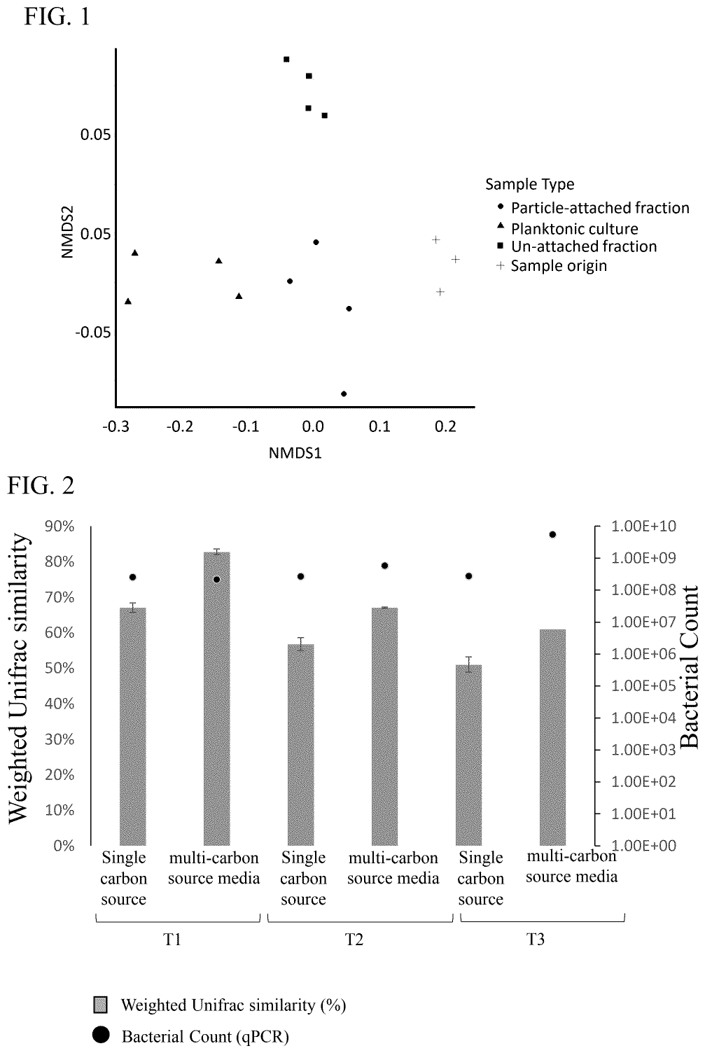

The results show (FIG. 1) that planktonic culture samples are more distant from the origin samples as compared to the distance of particle-attached bacterial fraction and un-attached fraction.

These results show that culturing in a presence of a particle yields compositions, comprising either planktonic and/or attached bacterial, having increased similarity compared to planktonic culturing (e.g., culturing in the absence of particle).

These results indicate that in order to obtain a composition with a high similarity to an origin sample, it is of advantageous to culture the plurality of bacteria in the presence of a particle.

Accordingly, unless indicated otherwise, in all the following examples, culturing was carried out in the presence of particles.

### Example 2

### The Effect of Carbon Source Type in the Media on Similarity to an Origin Sample and on Bacterial Load

In the following example the effect of different carbon sources on the ability to grow human-derived bacteria and produce a bacterial comprising composition having a high similarity to an origin fecal sample was assessed. In addition, the bacterial load was tested.

For this purpose, bacterial comprising composition were processed and cultured as elaborated above under the Material and Methods section in either a medium comprising a single carbon source (e.g., glucose) or a medium comprising a multi-carbon source. The multi-carbon source comprised Glucose; Maltose; Trehalose; and Starch. Each medium further comprised: Yeast extract; Peptone extract; Sodium Chloride; and Disodium hydrogen phosphate. Culturing was carried out in flasks (small scale).

Similarity was determined by using the Weighted Unifrac Similarity metric, and bacterial load was determined by qPCR measurements. Sampling was carried out from the particle-attached bacterial phase at different time points (referred to as T1, T2 and T3).

The results show (FIG. 2) that all tested groups had a similarity greater than 50% to the origin sample, and a bacterial load (qPCR) greater than 1·E⁸ per 1 gr particle. However, using a multi-carbon source medium resulted in a higher bacterial load while maintaining a higher similarity relative to single carbon source medium.

Therefore, the current study shows that advantageously, using more than one carbon source in the culturing media results in increased similarity to an origin sample.

### Example 3

### The Effect of Adding Microelements in the Media on Similarity to an Origin Sample

In the following example, the effect of adding microelements to a glucose medium or to a multi-carbon sources medium on the similarity of the composition to the origin sample was examined. The microelements used were Manganese source; Copper source; and Iron source. In addition, the bacterial load was tested.

For this purpose, compositions comprising bacteria were processed and cultured as elaborated above under the Material and Methods section. Culturing was carried out in either a medium comprising a single carbon source (glucose) or a medium comprising multi-carbon sources (Glucose; Maltose; Trehalose; and Starch) with or without supplementation of microelements. Each medium further comprised: Yeast extract; Peptone extract; Sodium Chloride; and Disodium hydrogen phosphate. Culturing was carried out in flasks (small scale).

Sampling was carried out at three time points (T1, T2 and T3) during culturing, and the Weighted Unifrac similarity and bacteria load (qPCR measurements) were evaluated. The reference sample is marked as “Origin Samples”.

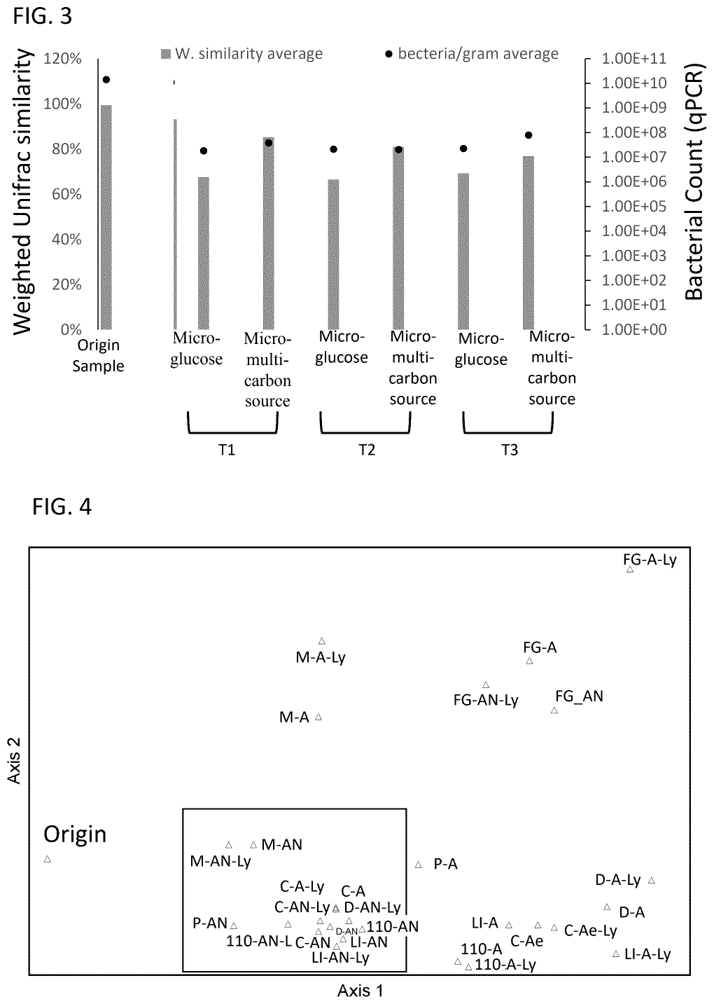

The results show that (FIG. 3) a similarity greater than 60% was obtained in all tested groups. Addition of microelements to the multi-carbon media resulted in a greater similarity of more than 70%. The results also showed a trend towards a greater bacterial load of the obtained co-culture when using a medium comprising both microelements and a multi-carbon source.

### Example 4

The Effect of Culturing Under Aerobic Vs. Anaerobic Conditions on Similarity Level to an Origin Fecal Sample

In the following example the effect of anaerobic or aerobic conditions on the bacterial similarity level was examined.

Bacterial samples were processed and cultured as described above under the Material and Methods section using different media substrates under anaerobic or aerobic conditions. The media tested were: Brain-Heart Infusion Medium (BHI) and various food-grade media compositions suitable for growing human gut bacteria. At the end of the culturing, the similarity (based on Bray-Curtis measure) was evaluated (origin Fecal Liquid sample is marked as “Origin”; anaerobic conditions are marked as AN).

The results presented as a PCoA (Metric Multidimensional scaling, MDS) plot (FIG. 4) show that employing anaerobic conditions during culturing was superior for maintaining similarity.

As anaerobic conditions were found to be superior in obtaining increased similarity, anaerobic conditions were employed in all the following examples.

### Example 5

### Similarity to a Fecal Origin Sample Using Different Media and Scale-Up Fermentation

In the following example, the effect of the media used and scale-up process on the similarity level was evaluated.

For this purpose, bacterial samples were processed and cultured as described above under the Material and Methods section using different source media substrates. Culturing was carried out in the presence of particles and under anaerobic conditions in either: small- or medium-scale volumes (50 ml-6 L). Table 1 below shows the conditions used during culturing.

For similarity and bacterial load evaluations, representative samples of particle-attached bacteria were collected from each treatment and the Weighted Similarity level of the bacterial population, and the bacterial count (based on qPCR measurements) were analyzed.

TABLE 1
Treatment Groups

| Treatment | |
|---|---|
| Group | Media |
| Fermenter* | Medium comprising Glucose; Yeast extract; |
| 1** | Peptone extract; Sodium Chloride; |
| | Disodium hydrogen phosphate (medium PG2) |
| 2** | Brain-Heart Infusion Medium (BHI)*** |

*Medium scale production.
**Small scale production.
***A standard commercial medium for growing fecal bacteria.

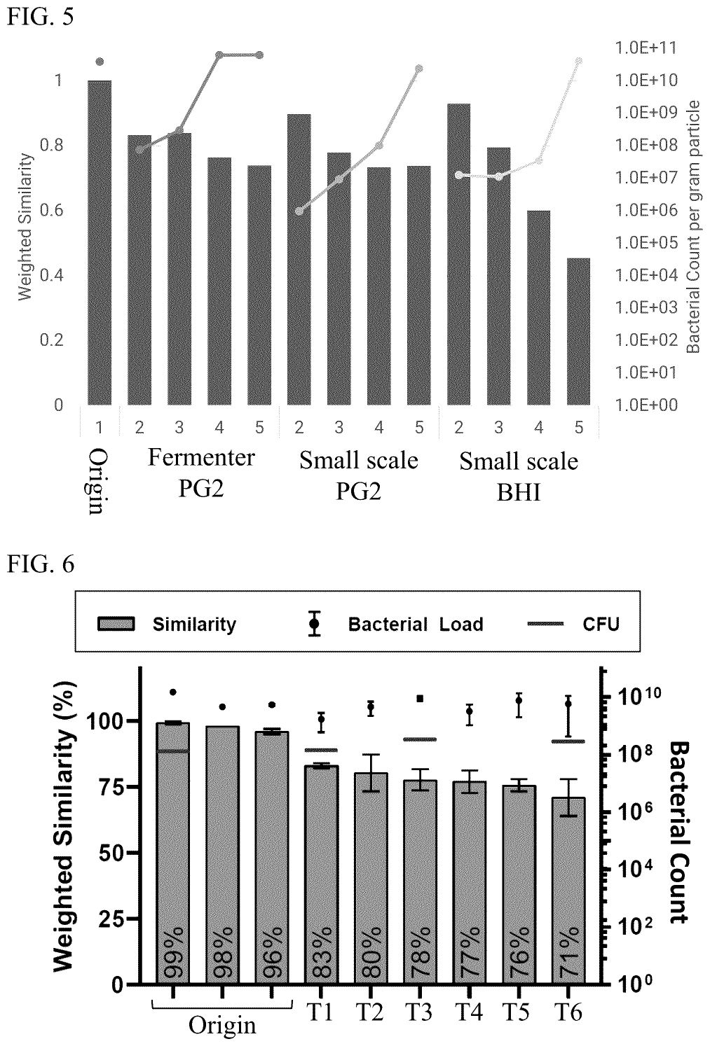

The results show (FIG. 5) that culturing in PG2 medium results with both an increased bacterial count and high similarity values, compared to culturing in a standard medium, e.g., BHI. The current results also show that culturing in PG2 medium advantageously maintained a high similarity level over a prolonged period of time.

When comparing fermentation with PG2 medium in small-scale vs. medium scale (Fermenter), the results show higher similarity values in a fermentation carried out in medium-scale. The bacterial counts in medium- and small-scale at timepoint 4 were above 6·E¹⁰ and 1·E⁸, respectively, illustrating the scalability of the process.

In another experiment, the inventors examined the number of days of culturing required so as to obtain a composition/co-culture having increased similarity greater than or equal to 50% to the origin sample, and a bacterial load of at least 1·E⁶ per gr. It was found that culturing for a period ranging between 6 hours and 6 days advantageously enabled obtaining both parameters (data not shown). When a multi-carbon source was used, a culturing period between 12 hours and 5 days resulted in a Weighted Unifrac similarity of greater than or equal to 70%, and a bacterial load of at least 1·E⁸ per gr.

Therefore, the method enables scalability and obtaining a composition similar to a fecal biological sample.

### Example 6

### Consistency of the Process Using Different Origin Samples

The following example aims to examine whether the method is capable of producing a composition having a high similarity to an origin sample although using different origin samples having different populations.

The results presented in FIG. 6 show combined data from 3 processes using 3 origin samples from the same donor collected on different days. The samples were processed and cultured independently as described above under the Material and Methods section in medium scale conditions (6 L) including a multi-carbon source medium comprising: Glucose; Maltose; Trehalose; Starch; Yeast extract; Peptone extract; Sodium Chloride; Disodium hydrogen phosphate; Manganese source; Copper source; and Iron source.

The results show the Weighted Unifrac similarity and bacterial load (qPCR & CFU measurements) at different time points (T1-T6) from the three independent cultures.

The results demonstrate that all time points resulted in a similarity of greater than 71% to a fecal origin sample (marked as “Origin”), with a relatively low SD, indicating the ability of the method to consistently yield a composition with high similarity to the origin sample. Also, the results show that the similarity level decreased with time while the CFU as well as bacterial count (qPCR) increased (e.g., compare T1 with T6).

This exemplifies that the provisions of the method according to an embodiment of the invention provides a composition with high similarity, and increased bacterial count even when using different origin samples varying in their microbiome profile.

In another experiment, the reproducibility and repeatability of the process was further evaluated.

For this purpose, five independent culturing processes carried out as described in the materials and methods, each using a different origin sample. The five independent processes were analyzed by comparing the produced composition and the respective origin sample in terms of observed species, observed genera, and weighted similarity parameter metric.

The observed genera in the origin samples were in the range of 42-58 (average of 50.1), while the composition contained 39-55 genera (average of 43.3; 84.4% genera richness as compared to the origin sample). The observed species in the origin samples were in the range of 131-196 (average of 168.1), while the composition contained 104-188 species (average of 138.3; 82.8% species richness as compared to the origin sample).

The average weighted similarity to the origin sample of the five experiments was 77.1% (each sample compared to its corresponding origin sample).

The inventors determined similarity using the Bray-Curtis values of the five experiments which was found to be in the range of 19-46% with an average of 35.1%. Hence, when using a similarity index that does not consider the phylogenetic relatedness among the bacterial populations such as Bray-Curtis, a lower value may be obtained as compared to the values obtained when using other similarity metrics.

The results show that although different origins were used, the compositions maintained a similarity higher than 77% to their origin samples while showing comparable numbers of observed species and genera.

These results show that regardless of the variation in origin sample community, the method advantageously enables to “copy” the origin sample and yield a synthetic composition with high similarity thereto.

Accordingly, the similarity analysis of the composition to an origin sample can be determined using various similarity metrics, however, some methods may result in a lower similarity value, e.g., as exemplified above.

The above Examples show the ability of different embodiments of the invention to obtain high similarity of bacterial populations while obtaining a high bacterial count. Examples 7 and 8 below show the ability of the method to further preserve other similarity features; e.g., similarity of other communities such as viral, fungal, in addition to bacterial community; and of metabolic routes/pathways.

### Example 7

### Similarity of Other Communities in a Composition Produced According to an Embodiment of the Invention to an Origin Sample

In the following example, the similarity of other communities in the composition were evaluated compared to an origin sample.

Fecal origin samples were processed and cultured as described above under the Material and Methods section, and the obtained composition were analyzed for their viral, fungal and bacterial communities using WGS. The similarity levels were measured using Bray Curtis similarity. The graphs are presented as non-metric multidimensional scaling (NMDS) of Bray-Curtis similarity metric performed, between the composition (square shape) and a reference fecal sample (diamond shape).

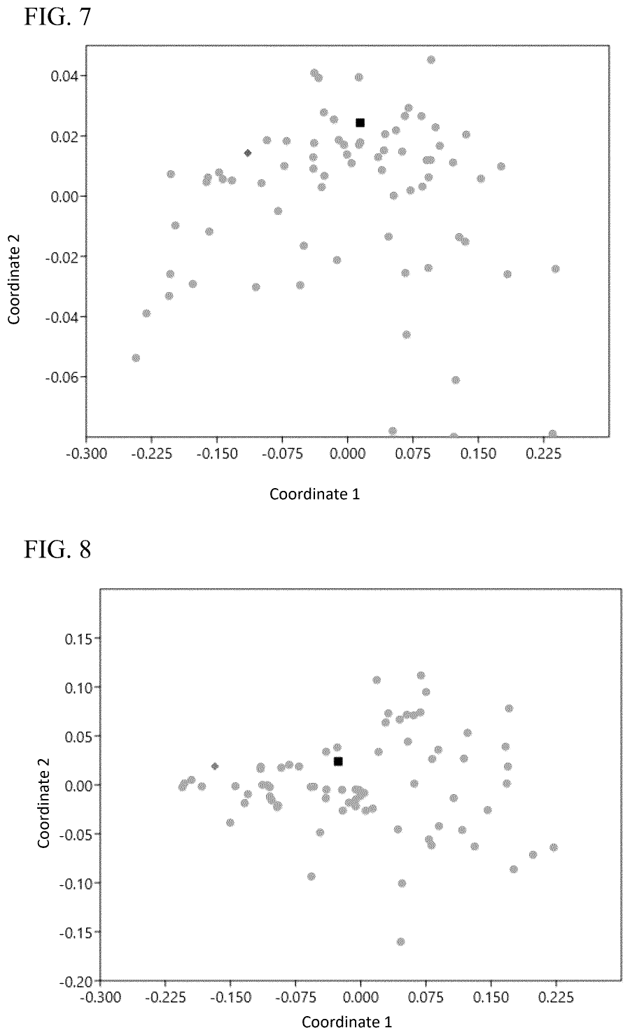

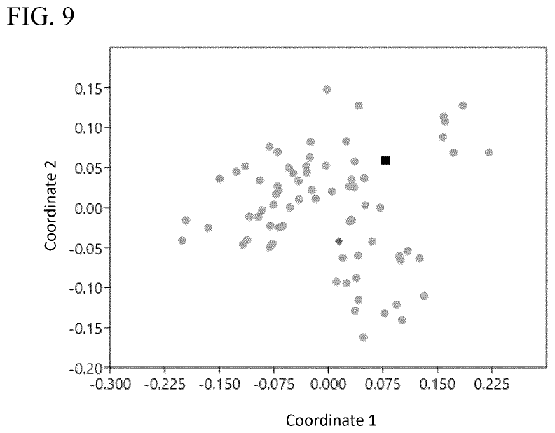

FIGS. 7-9 represent the similarities of the fungal, viral and bacterial communities, respectively. The similarity is observed by proximity of the composition to the origin sample in the plots.

The results show that a composition produced according to an embodiment of the invention preserved high fungal and viral similarity, and also corroborated the previous results showing that a high similarity of bacterial population is obtained. Additionally, the composition is in proximity to dozens other representative stool samples from healthy individuals. Specifically, the composition preserved 100% of the fungal species, 71.4% of bacterial viruses (genera) and 93.5% of bacterial species, identified in the original stool sample (Diversity Recovery).

The results show that the method advantageously enables to produce a composition having microorganism communities having a high similarity to an origin sample, thereby showing high potential as being used as a pharmaceutical composition in a similar manner as carrying out FMT treatments.

### Example 8

### Maintenance of Metabolic Routes in a Composition Produced According to an Embodiment of the Invention

In the following example, the ability of the method of the invention to preserve metabolic routes of an origin fecal sample were evaluated.

The ability to maintain metabolic routes/pathways, e.g., the potential to produce, synthesize, consume and/or utilize certain metabolites/organic compounds, was examined by a publicly available analysis software based on WGS data.

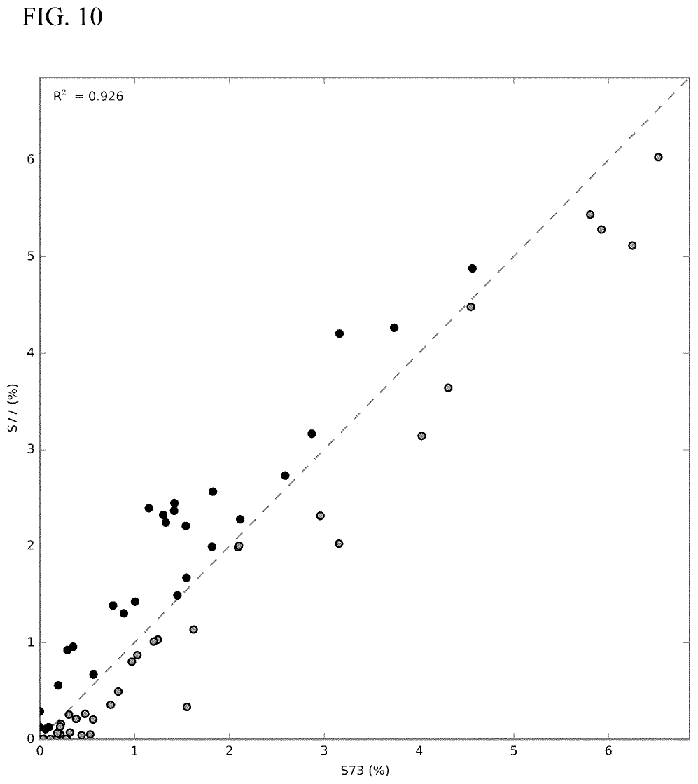

The results are presented in FIG. 10.

It can be seen that 104 (out of 108) metabolic super pathways were preserved in the composition (104 dots appear on the graph) with a R squared value of 0.926. The results show that the relative proportion among the pathways is substantially preserved.

This example shows that the method advantageously preserved the metabolic potential of an origin sample, in addition to the bacterial population and other microorganism that may be present in an origin sample.

As the method according to the invention advantageously enables to produce different compositions, e.g., (i) a composition comprising particle-attached bacteria, (ii) a composition comprising planktonic bacteria cultured in the presence of a particle, and (iii) a composition comprising both fractions, either as a mix of (i) and (ii) at any ratio or as combined fractions, Examples 9, 10 and 11 below show embodiment advantages of the various compositions.

### Example 9

### Different Compositions Produced According to the Invention

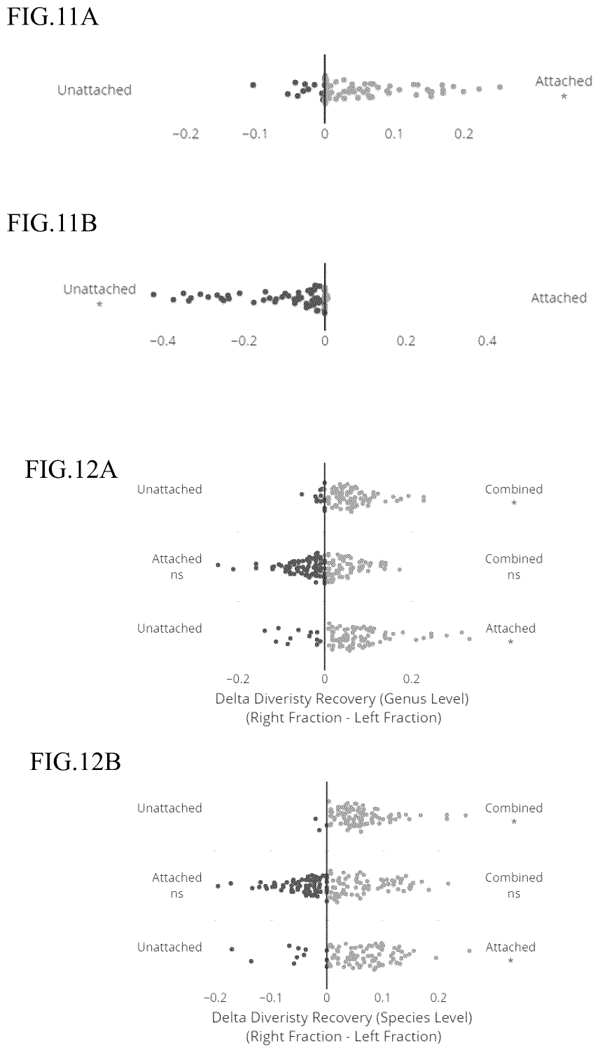

In the following example, the growth preference of the 10 most abundant bacteria in the tested composition sample (at family taxonomic level), i.e., preference to planktonic or attached growth form, was examined. The analysis was carried out by measuring the delta between the relative abundance of each bacterium in the particle-attached phase and the un-attached phase. For illustration two graphs are presented FIGS. 11A-11B.

The results show that out of the top ten most abundant bacteria, 3 bacterial families were more abundant in the attached fraction whereas 4 bacterial families were more abundant in the un-attached phase. Three additional bacterial families were found in similar relative abundance in both phases. Thus, the inventors demonstrated the potential role of the particles in maintaining the bacterial diversity in the composition similar to that of the origin sample.

Accordingly, the method of the invention advantageously enables to produce separated fractions, enriched with specific bacteria, that can be used as a standalone composition. The two separated compositions can also be mixed, at any desired ratio, to obtain a final composition enriched with specific bacteria of interest, e.g., having a positive correlation with amelioration, recovery or remission of a certain disease or condition.

Alternatively, by including both attached and un-attached phases, the presence/abundance and relative abundance of most taxonomic groups can be preserved following culturing, regardless of the bacteria phase preference, thereby maintaining the original eco-system of the origin sample.

### Example 10

### Similarity of Different Compositions Produced According to the Invention

Increased microbial diversity, as observed in healthy individuals, in known to restore the protective function of the microbiome against C. difficile. Accordingly, producing a microbial composition having a high microbial diversity similar to that of a fecal sample is of great importance.

In the following example, the diversity recovery was analyzed in different compositions produced according to the invention (i.e., a composition comprising particle-attached bacteria, a composition comprising un-attached fraction and a combined composition comprising both fractions). The bacterial diversity was measured in the genus and species level. In addition, the bacterial load was measured in each fraction by qPCR. In addition, weighted similarity was also evaluated in the different fractions.

FIGS. 12A-12B show the delta diversity recovery (at genus and species level, respectively) between two tested compositions.

The results show that the combined fraction and the particle attached fraction exhibited an increased diversity recovery of genera and species from the origin sample as compared to the un-attached fraction, demonstrating the potential role of the particles in maintaining the bacterial diversity in the composition similar to that of the origin sample. In addition, the inventors found that the combined fractions exhibited an increased bacterial load as compared to each fraction alone (data not presented).

Accordingly, combining both fractions can be of advantage, as compared to each separated composition, e.g., when a composition having an increased diversity recovery and an increased bacterial load is desired. In addition, it was also found that the combined composition had an increased weighted similarity as compared to each of the fraction alone.

### Example 11

### Relative Abundance of Different Compositions Produced According to the Invention

In another analysis, the proportions (relative abundance) between different taxonomic bacterial groups (at the family level) were compared between different fermentation samples/compositions (particle-attached phase, un-attached phase and a combined composition). The obtained proportion in each composition was compared to the proportion in the respective origin sample. The data were obtained from multiple experiments using different origin samples.

In addition, the proportions were also evaluated in samples obtained from planktonic culturing (culturing in the absence of particles) carried out under similar culturing conditions to that of culturing in the presence of particles. The proportions are presented in Table 2 below.

The bacterial groups selected for the analysis were among the most abundant bacteria present in the tested samples. Additionally, these bacterial groups were reported in the literature as potential producers of short chain fatty acids (SCFA) and bile acids converters, which correlated with a positive treatment outcome of various diseases or conditions such as C. diff infection, atopic dermatitis and ulcerative colitis.

TABLE 2
Proportions between different taxonomic groups of bacteria
in different samples and in an origin sample

| | | | Statistical |
|---|---|---|---|
| | | Median | significance |
| | | value of | to reference |
| | Sample type | the ratio | fecal sample |
| Family A:Family D | Reference fecal sample | 1.4 | — |
| | Attached fraction | 1.3 | NS |
| | Un-attached fraction | 1.9 | p < 0.001 |
| | Planktonic culturing | 0.4 | p < 0.05 |
| | Combined | 1.4 | NS |
| Family C:Family B | Reference fecal sample | 1 | — |
| | Attached fraction | 0.1 | p < 0.001 |
| | Un-attached fraction | 0.8 | NS |
| | Planktonic culturing | 0.4 | NS |
| | Combined | 0.9 | NS |
| Family A:Family B | Reference fecal sample | 9.4 | — |
| | Attached fraction | 1.3 | p < 0.001 |
| | Un-attached fraction | 2.2 | p < 0.001 |
| | Planktonic culturing | 0.2 | p < 0.05 |
| | Combined | 3 | p < 0.001 |
| Family D:Family B | Reference fecal sample | 6 | — |
| | Attached fraction | 1 | p < 0.001 |
| | Un-attached fraction | 1 | p < 0.001 |
| | Planktonic culturing | 0.5 | p < 0.05 |
| | Combined | 1.7 | p < 0.001 |
| Family D:Family C | Reference fecal sample | 8.5 | — |
| | Attached fraction | 6.2 | p < 0.01 |
| | Un-attached fraction | 0.8 | p < 0.001 |
| | Planktonic culturing | 0.8 | p < 0.05 |
| | Combined | 1.8 | p < 0.001 |
| Family B:Family E | Reference fecal sample | 1.2 | — |
| | Attached fraction | 4.7 | p < 0.001 |
| | Un-attached fraction | 2.6 | p < 0.001 |
| | Planktonic culturing | 10.9 | p < 0.05 |
| | Combined | 2.4 | p < 0.001 |
| Family A:Family E | Reference fecal sample | 17 | — |
| | Attached fraction | 5.7 | p < 0.001 |
| | Un-attached fraction | 6 | p < 0.001 |
| | Planktonic culturing | 2.8 | p < 0.05 |
| | Combined | 7 | p < 0.001 |

NS—Non-significant difference

These results show that in most cases to better resemble the origin sample and preserve the proportions between different bacterial families, it is required/of advantage to combine the two fractions to a final composition.

Importantly, in most cases, the un-attached phase (produced according to the methods described hereinabove) showed a lower deviation from the tested proportions in the origin sample compared to the proportions in planktonic culturing (i.e., culturing without particles). Thus, culturing in the presence of particles surprisingly facilitated preservation of the tested proportions.

These results also corroborate the previous results and show that in order to obtain a composition with a high similarity to an origin sample, it is required/of advantage to culture the plurality of bacteria in the presence of a particle.

### Example 12

### Culturing a Pooled Origin Sample in One Vessel

In the following example, the advantage of using a pooled origin sample derived from more than one origin, e.g., donor, as a staring material in a method according to an embodiment of the invention was examined.

For this purpose, several characteristics were evaluated: (i) various α-diversity parameters in both the pooled origin and produced composition vs. the separated samples and their respective produced composition; and (ii) the ability of a culturing method according to an embodiment of the invention to preserve difficult-to-culture bacteria when using the pooled samples as compared to using a single source origin. Three different origins were collected in this experiment.

Table 3 below show the α-diversity parameters in the two different origins and in a composition prepared therefrom.

Surprisingly, it can be seen that a pooled origin sample, comprising various microorganism populations characterized as having differing culturing requirements, resulted in a composition having increased α-diversity parameters as compared to a composition produced from one source.

TABLE 3
α-diversity parameters in single origin and pooled
origins and in compositions prepared from such origins

| | Single Origin* | | Mix of Origins | |
|---|---|---|---|---|
| Tested Parameter | Origin | Composition | Origin | Composition |
| Observed genera | 50.0 | 35.9 | 58.0 | 40.0 |
| Chao1 | 170.9 | 114.4 | 212.7 | 126.0 |
| Observed species | 157.8 | 104.3 | 196.0 | 116.0 |
| Shannon | 5.7 | 4.1 | 6.3 | 5.0 |
| Simpson Reciprocal | 27.9 | 8.2 | 45.8 | 13.8 |

*Represents the average of the three different origins that were used for the pooled origin sample.

Additionally, the ability of the present culturing method to produce a composition that preserves difficult-to-culture bacteria was examined. As a representative genus, the relative abundance of the bacteria Akkermansia was evaluated.

According to the literature, Akkermansia is a promising next generation probiotic and a difficult-to-culture bacteria in a complex bacterial population (Derrien, M., Vaughan, E. E., Plugge, C. M. and de Vos, W. M., 2004. Akkermansia muciniphila gen. nov., sp. nov., a human intestinal mucin-degrading bacterium. International journal of systematic and evolutionary microbiology, 54(5), pp. 1469-1476).

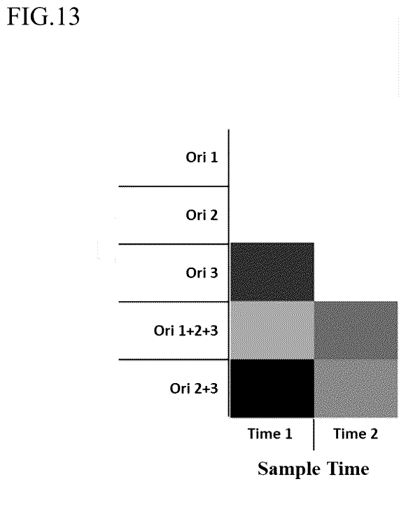

FIG. 13 shows that origin sample 3 contained Akkermansia at the time point 1 of culturing, however the tested bacterium was not preserved in the composition at the time point 2. It can be seen that pooling of several origin samples resulted in preservation of Akkermansia at time point 2 of the cultivation.

Moreover, pooled samples also improved preservation of other potentially beneficial genera, such as Roseburia, Lachnoclostridium-Roseburia, Fusicatenibacter, Coprococcus, Ruminiclostridium (data not shown).

Thus, advantageously, using pooled samples as the starting material according to an embodiment in the method of the invention (vs. using a single origin sample), can provide conditions that enable preservation of various bacterial genera.

Advantageously, the method enables to produce a composition having a high similarity to an origin sample that can potentially be used as a pharmaceutical composition as exemplified herein below. The culturing system according to the invention advantageously resembles a human gut microbiota and can be used, inter-alia, as an in-vitro model for assessing the effect of a compound on a subject as exemplified herein below.

### Example 13

A Co-Culture Comprising a Plurality of Bacteria Originated from a Saliva Sample, Cultured in One Vessel in the Presence of Particles and Under Anaerobic Conditions

The ability of growing saliva bacterial population according to an embodiment of the invention from a saliva sample was examined in this example.

Saliva was grown in small flasks using a medium comprising: multi-carbon source, Yeast extract, Peptone extract, Sodium Chloride, Disodium hydrogen phosphate, microelements and particles, as described above in the Materials and Methods section.

The flasks were incubated in the anaerobic chamber and sampled throughout the fermentation process. The achieved CFU/ml was about 1·E⁸.

The results demonstrated that the weighted similarity was higher than 50%, indicating that the method according to the invention is suitable for culturing plurality of bacteria originated from a saliva sample (data not shown).

### Non-Limiting Examples of Utilizing a System/Composition According to an Embodiment of the Invention

The below experiments exemplify that an in-vitro system/model generated according to an embodiment of the invention can be effectively used, e.g., as a gastrointestinal model, in order to determine the effect of a compound on a subject's microbiome; and that different microbiota samples can be used to establish the model. Also, the experiments exemplify that the system can be used for generating different microbial profiles.

As an example for the effectiveness and reproducibility of the system in evaluating the effect of a compound on a plurality of bacteria, different compounds or known drugs were examined.

Unless indicated otherwise, the compounds were added to the in-vitro model system during inoculation. The effect of the compounds on the plurality of bacteria was examined in the particle-attached and un-attached bacteria phases. Compound_A, A′ and D were added at the therapeutically used concentration and in increasing concentrations.

In all the experiments below, culturing was carried out in a multi-carbon source medium (Glucose; Maltose; Trehalose; Starch) further comprising a combination of microelements (Manganese source; Copper source; Iron source); Yeast extract; Peptone extract; Sodium Chloride; and Disodium hydrogen phosphate.

### Example 14

### Microbiome Profile of Different Samples

In the following example the ability to generate different systems/models that comprise a similarity to different origin samples having different microbiota population profiles was examined.

In addition, the example presents verification of the ability of the system to maintain a similarity to the initial sample also in small-scale conditions; and examination of the sensitivity of the system to identify changes in the microbiome profile of a subject, by culturing samples obtained from each donor on three different days (i.e., 3 independent experiments carried out by obtaining 3 samples from Donor 1).

For the above purposes, a microbiota population comprising a plurality of bacteria was obtained from four different donors and 4 systems were generated (FIG. 17); Donor #one provided origin samples at three different independent time points; i.e., several systems were generated simultaneously: 4 systems from 4 different donors; and additional 2 systems generated from Exp. 1 & 2 of Donor #1 (altogether 6 in-vitro culturing systems). Several samplings were carried out from the particle-attached bacteria phase.

β-diversity (Bray-Curtis) of the different samples obtained from the 6 different culturing systems was examined and is presented in a non-metric multidimensional scaling (NMDS) plot.

The results (FIG. 17) show that each of the 4 donors was located at a distance from the others on the plot, and that different samples taken from the same donor at different time points throughout the culturing period, were located in the same area in the plot. These results indicate that different systems can be generated according to the donor's unique initial microbial pattern, and that the system can maintain the unique initial microbial pattern for a prolonged period of time.

When examining the β-diversity of the microbiome population in a system generated from three different origin samples from the same donor at the three sequential days, all the samples were located at the same region on the plot with a slight variation from each other. This indicates that the system can identify slight/subtle changes in the microbiome profile.

### Example 15

The Effect of Compound_A on the Bacterial Load of Different Culturing Systems Generated from Different Samples Derived from Different Donors

The previous example shows that different systems generated from different origin samples and different donors, resulted in a different microbiota population profile.

To examine the ability of the system to accurately determine the effect of a compound on a plurality of bacteria regardless of the different microbiome profile, Compound_A was contacted with 4 fecal samples from 4 donors. Following contact between the fecal sample and the particles, the compound was added into the different culturing systems and incubated with the bacterial population during the culturing period.

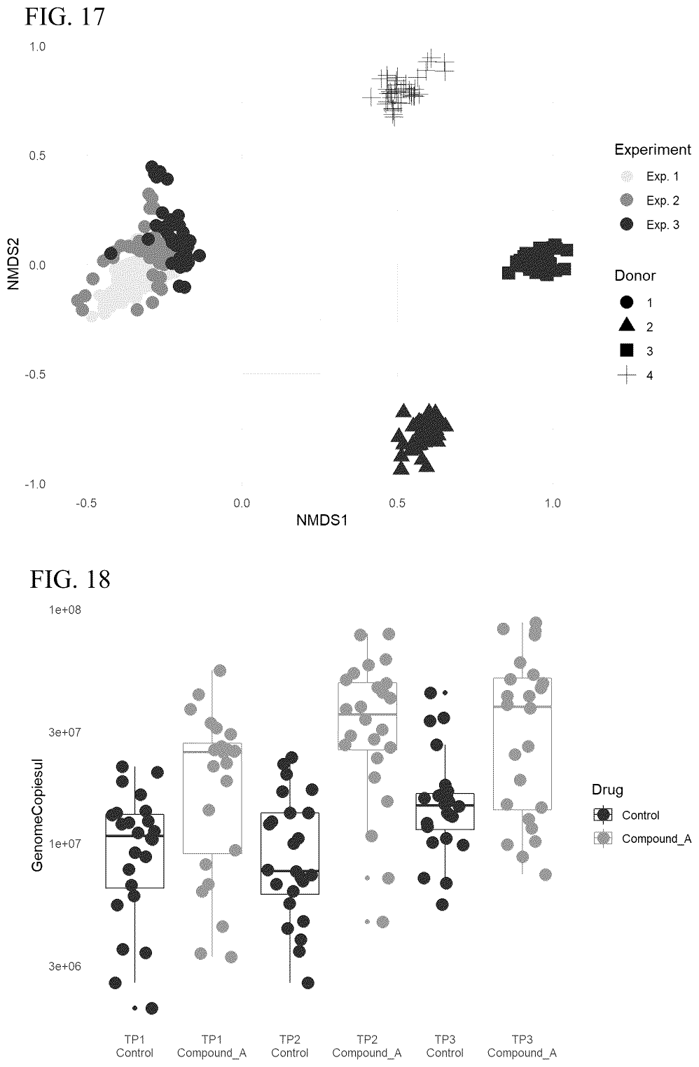

The effect of the compound on the bacterial count (based on qPCR measurement) was examined in the different generated systems at three time points (marked as TP1; TP2 and TP3) and is shown in FIG. 18.

FIG. 18 shows that addition of Compound_A resulted in an increase in the bacterial count at the three tested time points in all 4 generated systems (all presented on the same plot) compared to the untreated control system, indicating that the system can identify a common effect of a compound on the microbial population regardless of the initial bacterial profile of the system.

### Example 16

The Effect of Compound_A on the Microbial Profile of Different Culturing Systems Generated from Different Samples

To better define the ability of the system to examine the effect of a compound on the microbiome profile and identify changes in the microbiome community, Compound_A was added into 4 different systems (generated from 4 fecal samples obtained from 4 different donors) and the α- and β-diversity were examined. The compound was added during system culturing as indicated in the previous Examples, and sampling was carried out at three different time points (TP1, TP2 and TP3).

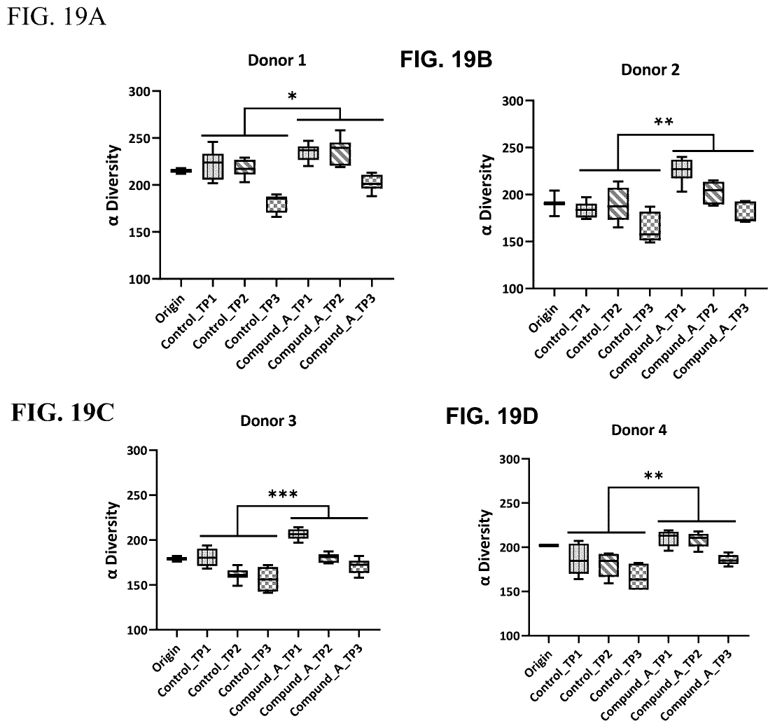

The α-diversity in each system is shown in FIGS. 19A-19D. Each figure shows the α-diversity of the sample before culturing, and at 3 time points during the culturing period of an untreated control system and a system treated with Compound_A. At each time point, the average value of TP1, TP2 & TP3 was calculated and used for assessment of the effect of the compound.

The results show that addition of Compound_A into each of the systems resulted in a significant increase in α-diversity compared to the untreated control bacterial population, regardless of the origin sample.

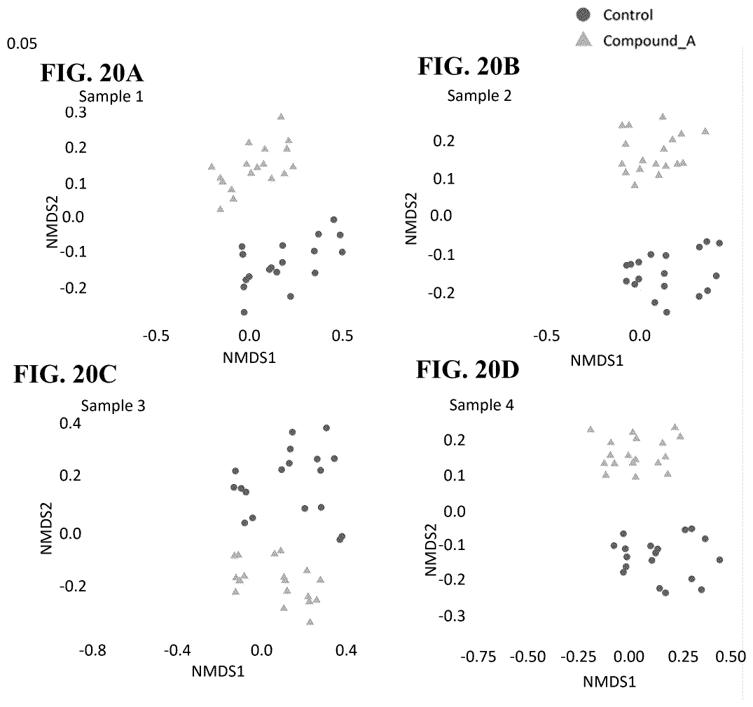

β-diversity of each system is shown in FIGS. 20A-20D, each figure shows the diversity at 3 time points during the culturing period of an untreated control system and a system treated with Compound_A (all time points are presented on the same plot).

The results show that exposure of a bacterial population to the tested compound modulated/altered the bacterial population, when compared to the bacterial population of the untreated control.

These results indicate that a system as exemplified herein according to an embodiment of the invention can identify an increase in the number of species and any change in the similarity level as a result of an exposure to a compound.

### Example 17

### The Effect of Compound_A on Modulation of Specific Bacterial Genera

The previous example shows that the culturing system according to an embodiment of the invention is an efficient tool, capable of identifying a common effect or a trend of a compound on the bacterial load and/or on the microbiome profile, regardless of the initial microbial profile of the origin sample/donor.

The aim of the following example is to use the data extracted from the system (e.g., bacterial load, α-diversity and/or β-diversity) and further investigate and identify bacteria modulated following exposure of the bacterial population to the tested compound. As exemplified above, bacterial modulation can be seen by a change in the positioning and/or distribution of the dots of a treated system vs. an untreated-system on an NMDS plot.

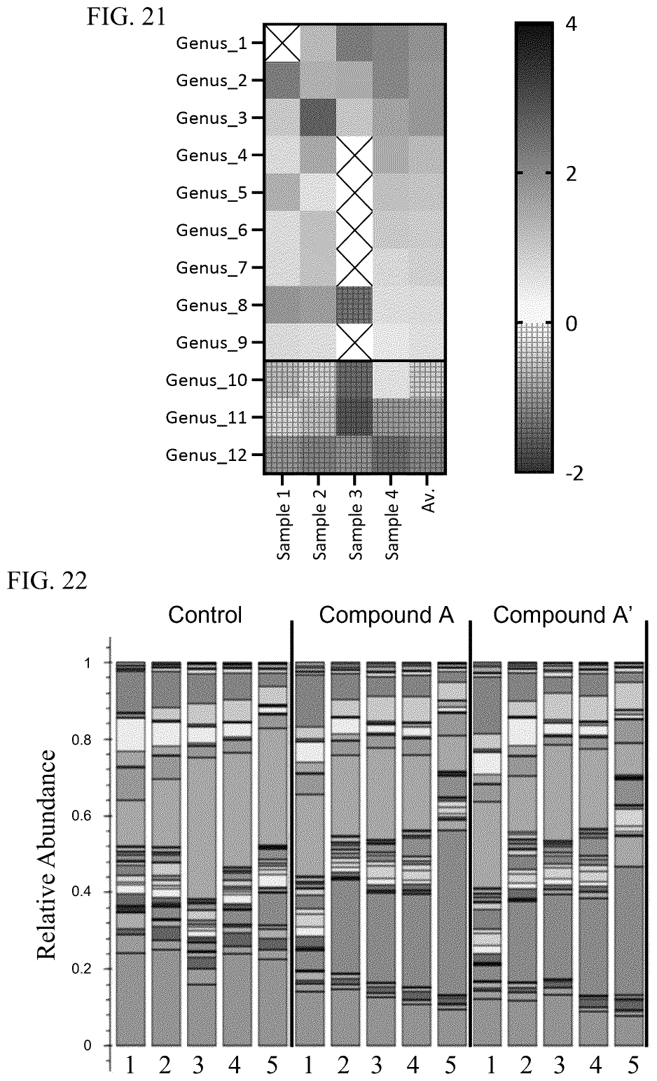

A heatmap showing different bacterial genera that were affected by exposure of a bacterial population to Compound_A is shown in FIG. 21. Each experiment was carried out in 4 treated systems simultaneously (Samples 1-4). Analysis was carried out as compared to an untreated control bacterial population.

In these experiments, a change in the genus level was considered as significant if the same trend (elevation or decrease) was observed in 3 out of the 4 tested samples (i.e., 75% of the samples showed an identical trend). The results show that 9 genera increased and 3 decreased. Similar trends were observed when comparing the biomarkers identified by the culturing system with biomarkers identified in a clinical trial carried out using the same compound.

Advantageously, examining the effect of a compound on a microbiome population in a culturing system according to an embodiment of the invention, simulating a flora of a subject, can provide insight to bacterial population modulation in view of exposure to such compound. Identifying modulated bacterial genus (or a modulation in other any taxonomic level) can be a therapeutic target for increasing the efficacy of the drug, e.g., by administering to a subject a drug-probiotic or a drug-prebiotic combination treatment aimed to restore/preserve the original or a healthier microbiome profile.

### Example 18

### Use of a System According to an Embodiment of the Invention as a High Throughput Screening Assay and as a Platform for Generating Different Microbial Profiles

In the following example, the effect of two compounds, Compound_A and a derivative thereof (Compound_A′), on the relative abundance was compared.

The two compounds were added into two different systems simultaneously under the same conditions, and sampling was carried out at 5 time points throughout the culturing period for relative abundance measurements. The results were compared to an untreated control system sampled on the same time points (altogether 3 systems were generated from the same original sample/donor).

The results (FIG. 22) show that the relative abundance of genera in all 5 time points was altered following treatment with either Compound_A or Compound_A′ relative to the untreated system. It can also be seen that the two treated groups exhibited a different microbial profile.

The results emphasize the sensitivity and ability of the system and method to be used in several single vessels simultaneously as a high throughput screening assay for comparing the effect of different treatments, e.g., different drug derivatives, on a subject's microbiome population; it is also demonstrated that the system can be used to generate different microbial profiles.

### Example 19

### Sensitivity of the Culturing System to Different Compounds

In another experiment, the ability and sensitivity of the system to identify changes in the microbiome profile and bacterial count in response to different chemical compounds, Compound_D and Compound_J, was evaluated.

For this purpose, each compound was added into a separate system both generated from the same origin sample, and similarity parameters and bacterial count were evaluated. Sampling was carried out at different time points from different phases as elaborated below.

Table 4 shows the effect of the compounds on the α diversity and the bacterial count.

TABLE 4
Compounds influence on richness and bacterial count

| | Sampling | | |
|---|---|---|---|
| | time | α diversity* | |
| Compound | point | (Richness %) | Bacterial count** |
| D | TP1 | 41.67 | 0.015 |
| D | TP2 | 18.52 | 0.510 |
| D | TP3 | 23.26 | 0.933 |
| J | TP2 | 113.80 | 7.450 |
| J | TP3 | 112.44 | 1.708 |

*Percentage change compared to the value of the relative untreated particle-attached bacteria fraction.
**Fold-change compared to the count of the untreated combined fractions. Bacterial count in each sample was measured by CFU.

Exposure of the bacteria to Compound_D resulted in a significant reduction in the bacterial count followed by a gradual increase to a level close to the level of the untreated fraction. In comparison, exposure of the bacteria to Compound_J, resulted in a 7.450-fold increase in the bacterial count, followed by a reduction.

α-diversity decreased in response to exposure to Compound_D, whereas exposure to Compound_J resulted in a minor increase in richness.

Notably, in both tested parameters the effects observed following exposure to Compound_D are similar to the effects known in the art.

Accordingly, these results corroborate the previous results and demonstrates the effectiveness and sensitivity of the system.

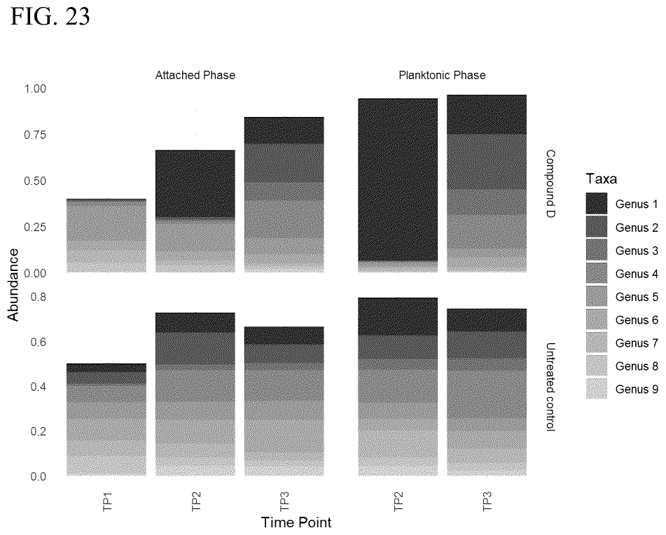

Next, the ability to evaluate the effect of a compound using particle-attached and un-attached fractions. For this purpose, the relative abundance of 9 specific genera was measured in both fractions following contact of the bacteria with Compound_D. The results are presented in FIG. 23.

Generally, the results show that the microbial profile of each fraction was differently affected following exposure to the compound. For example, the relative abundance of Genus 1 in response to Compound_D in TP3 resulted in 36.25% and 87.70% in the particle-attached bacteria and un-attached bacteria, respectively (see FIG. 23; upper panel).

The results demonstrate the significance of evaluating the effect of an added compound on the microbial population in both fractions, either combined and/or separated. Moreover, the results corroborate our previous results, showing the tendency of a genus to adhere to the particles and/or to prefer planktonic form growth. Notably, tendency of a specific bacterium to attach to particles, may imply on the potential for engraftment at the target site.

In parallel to the sensitivity experiment, the ability of a ‘stabilized’ or an ‘initial’ system to display changes in the microbial profile and bacterial count was evaluated by adding Compound_D, Compound_F and Compound_J, at two different time points. ‘Initial’—when the compound was contacted with the plurality of bacteria during the inoculation stage, prior to addition of particle into the system; and ‘stabilized’—when the compound was added after contacting the plurality of bacteria with particles and growth medium addition. These conditions may represent different physiological conditions of a subject, e.g., a more stable healthy microbiome vs. a dysbiotic microbiome. The α-diversity and bacterial count results are presented in Table 5.

TABLE 5
The effects of various compounds on richness and bacterial load

| | System | | |
|---|---|---|---|
| | condition at | | |
| | addition of | α diversity* | Bacterial |
| Compound | compound | (Richness %) | count** |
| D | Initial | 23.26 | 0.933 |
| D | Stabilized | 90.70 | 0.749 |
| F | Initial | 51.04 | 2.330 |
| F | Stabilized | 110.08 | 1.505 |
| J | Initial | 112.44 | 1.703 |
| J | Stabilized | 96.53 | 1.125 |

*Percentage change compared to the value of the relative untreated particle-attached bacteria fraction.
**Fold-change compared to the count of the untreated combined fractions. Bacterial count in each sample was measured by CFU.

The results show that exposure of bacteria to Compound_D and F at an ‘initial’ stage significantly reduced the α-diversity similarity level compared to the exposure of the bacteria to a ‘stabilized system’, however exposure of the bacteria to Compound_D resulted in a similar effect at both stages.

The α- and β-diversity parameters following exposure to several additional compounds at an ‘initial’ or ‘stabilized’ conditions corroborated the results and showed that the ‘stabilized system’ was less susceptible to changes in the bacterial population as compared to an ‘initial system’.

These results show that it is of advantage to evaluate the effect of the compound during different stages of the method.

### Example 20

Use of a System According to an Embodiment of the Invention for Producing a Composition Enriched with Bacteria Attached to a Particle by Addition of a Compound

In the following Example, the use of the system to enhance a specific bacterial growth form, i.e., attached to particles or being in planktonic growth form, was examined.

For this purpose, Compound_B was added into a culturing system prepared as described above and incubated with the bacterial population during the culturing period (Vessel 2). The compound was added following contact of the plurality of bacteria with the particles. The effect of the compound on the growth preference of 4 selected bacteria was examined by measuring their relative abundance in the entire bacteria population in both the attached and the plankton phases. The results were compared to an untreated control system (Vessel 1).

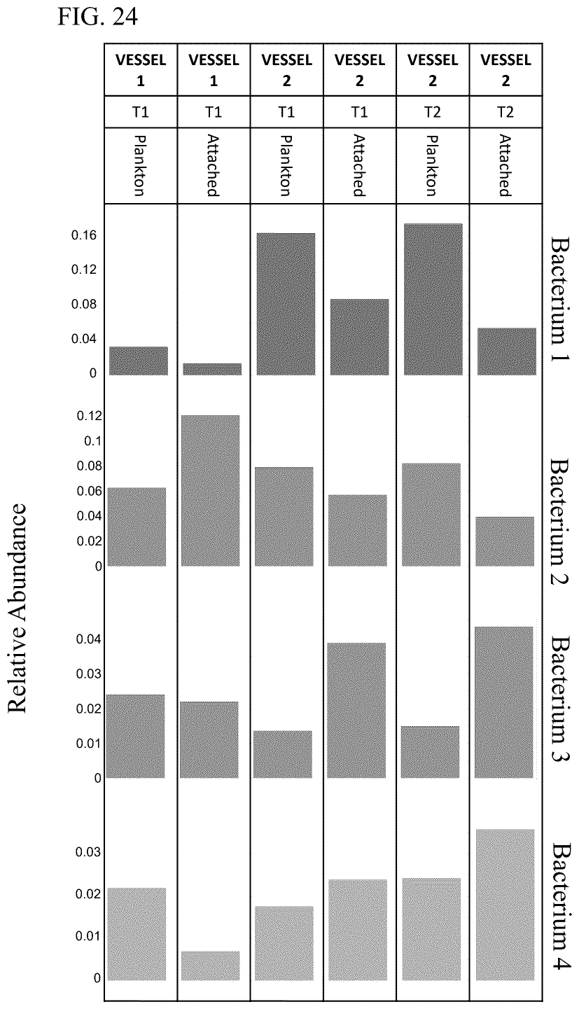

FIG. 24 shows that the relative abundance of Bacterium 1 was increased in view of exposure to Compund_B, whereas no difference was observed in the preferred growth form (tendency to planktonic growth). For Bacteria 2 and 4—addition of Compund_B resulted in a change in the growth form. Bacterium 2 demonstrated a switch in preference from attached form to planktonic form, whereas the opposite trend was displayed by Bacterium 4. For Bacterium 3, addition of Compound_B resulted in preference for attached growth form, whereas in the untreated system, a substantially similar relative abundance was observed in both fractions.

These results suggest that the system according to the invention can be used for identifying a compound capable of enhancing a specific bacterial growth form of a particular bacterium within a plurality of bacterial population, and for producing a composition enriched with a specific bacterium in an attached form and/or un-attached form.

Although the invention has been described in conjunction with specific embodiments thereof, it is evident that many alternatives, modifications, and variations will be apparent to those skilled in the art. Accordingly, it is intended to embrace all such alternatives, modifications and variations that fall within the spirit and broad scope of the appended claims.

1. A method for producing a composition comprising a co-culture comprising a plurality of bacteria grown in the presence of a particle, the co-culture comprises: i) at least 30% similarity to an origin sample, and ii) a bacterial load of at least 1·E⁴ per 1 gr particle, the method comprising:
providing microorganisms comprising a plurality of bacteria being derived from an origin sample;
contacting said plurality of bacteria with a particle, and allowing said plurality of bacteria to at least partially attach to said particle; and
culturing said plurality of bacteria at least partially attached to said particle in a growth medium for a period of less than 14 days,
wherein said culturing comprises culturing under anaerobic conditions,
thereby producing the composition comprising a co-culture comprising a plurality of bacteria grown in the presence of a particle comprising: i) at least 30% similarity to an origin sample; and ii) a bacterial load of at least 1·E⁴ per 1 gr particle.

2. The method of claim 1, wherein said plurality of bacteria are characterized as having differing growth, cultivation and/or proliferative conditions selected from the group consisting of: metabolic requirements, nutritional requirements, pH, Temperature, aerobic, obligatory anaerobic, facultative anaerobic, microaerophilic, attached form, planktonic, growth medium, flow, shake, stir, agitation, static, moist, low-humidity, and any combination thereof and optionally wherein said culturing is carried out until said co-culture reaches a similarity greater than or equal to 30% to said origin sample, and a bacterial load of at least 1·E⁴ per 1 gr particle.

3. (canceled)

4. The method of claim 1, wherein said culturing period ranges from 6 hours to 14 days.

5. The method of claim 1, wherein said co-culture comprises at least 30% similarity to said origin sample when said similarity is determined by a metric that considers the genetic relatedness of the bacteria using any one of: next generation sequencing (NGS) technology, whole genome sequencing (WGS), or both.

6. The method of claim 1, wherein said similarity comprises at least 50% Weighted Unifrac similarity.

7. The method of claim 1, wherein said similarity comprises at least 70% Weighted Unifrac similarity, and said co-culture comprises a bacterial load of at least 1·E⁸ per 1 gr particle.

8. The method of claim 1, wherein said similarity between said co-culture and said origin sample comprises similarity between bacterial populations.

9. The method of claim 1, wherein said origin sample further comprises additional microorganisms comprising any one of: archaea, viruses, fungi, or any combination thereof, optionally wherein said co-culture further comprises said additional microorganisms, and optionally wherein said similarity between said co-culture and said origin sample further comprises similarity of at least one of: archaea, viruses, fungi populations, or any combination thereof.

10.-11. (canceled)

12. The method of claim 1, wherein said provided plurality of bacteria belong to at least 5 species of bacteria and/or at least 2 bacterial genera.

13. The method of claim 1, wherein said composition comprises bacteria in planktonic form and bacteria at least partially attached to said particles.

14. The method of claim 1, wherein said growth medium comprises at least two carbon sources.

15. The method of claim 1, wherein said growth medium comprises carbon sources from at least two chemical groups being selected from the group consisting of: monosaccharide, di-saccharide, polysaccharide and any combination thereof.

16. The method of claim 1, wherein said growth medium comprises at least one monosaccharide, at least one disaccharide and at least one polysaccharide.

17. The method of claim 1, wherein any one of said contacting step, said culturing step, or both, is carried out in a single vessel.

18. The method of claim 1, further comprising a step of separating bacteria un-attached to said particle from bacteria attached to said particle at at-least one time point selected from the group consisting of: prior to, during, after the culturing step, and any combination thereof, thereby producing: (i) a composition comprising bacteria in planktonic form; and/or (ii) a composition comprising bacteria attached to said particle.

19. The method of claim 1, wherein said plurality of bacteria are provided within a vessel, and wherein said method further comprises the steps of: adding at least one compound to said vessel; and determining any feature selected from: (i) bacterial diversity; (ii) bacterial relative abundance; (iii) bacterial load; (iv) any other effect of said at least one compound on said plurality of bacteria; (v) any change in said at least one compound, and (vi) any combination of (i) to (v);
thereby evaluating the effect of: at least one compound on a plurality of bacteria, a plurality of bacteria on at least one compound, or both.

20. The method according to claim 1, wherein said origin sample is selected from the group consisting of: derived from at least one origin, derived from at least one subject, is a microbiome sample, a skin sample, an oral sample, a fecal sample, a vaginal sample, comprises gut microbiota, and any combination thereof.

21. The method of claim 19, wherein said compound modifies: (i) said similarity level of at least 30% at the end of the culturing period, (ii) said bacterial load of at least 1·E⁴ per 1 gr particle at the end of the culturing step, or both (i) and (ii), and optionally wherein modifies comprises: increasing or reducing any one of said similarity level, said bacterial load, or both.

22. (canceled)

23. The method of claim 19, wherein any one of: (i) said method is carried out simultaneously in several single vessels; said bacterial diversity; said bacterial relative abundance; said bacterial load; and/or said other effect of said at least one compound on said plurality of bacteria is compared to the corresponding feature in the plurality of bacteria of said origin sample, and wherein modification in said feature is indicative that said at least one compound has an effect on said plurality of bacteria; (ii) said method further comprises culturing a control plurality of bacteria that is not exposed to said compound in a separate vessel, and wherein said bacterial diversity; said bacterial relative abundance; said bacterial load; and/or said other effect of said at least one compound on said plurality of bacteria is compared to the corresponding feature in said control plurality of bacteria, and wherein any modification in said feature is indicative that said compound has an effect on said plurality of bacteria; (iii) wherein said adding and/or determining is carried out using bacteria attached to said particle, bacteria un-attached to said particle, or both; and (iv) any combination of (i) to (iii).

24.-26. (canceled)

27. A composition produced according to the method of claim 1, and optionally further comprising an acceptable carrier or excipient.

28.-30. (canceled)
